(19) United States
(12) Patent Application Publication
(10) Pub. No.: US 2009/0136521 A1
(43) Pub. Date: May 28, 2009

(54) Method for Selectively Depleting Hypoxic Cells

(75) Inventors: Kalindi Parmar, Arlington, MA (US); Peter Mauch, Boston, MA (US); Julian Down, Cambridge, MA (US)

Correspondence Address: LAHIVE & COCKFIELD, LLP;FLOOR 30, SUITE 3000, ONE POST OFFICE SQUARE, BOSTON, MA, 02109, US

(73) Assignees: Genetix Pharmaceuticals , Inc., Cambridge, MA (US); Dana-Farber Cancer Institute , Inc., Boston, MA (US)

(21) Appl. No.: 11/992,898

(22) PCT Filed: Sep. 29, 2006
(86) PCT No.: PCT/US2006/038553

§ 371 Date: Mar. 31, 2008

Related U.S. Application Data
(60) Provisional application No. 60/723,183, filed on Oct. 3, 2005.

(51) Int. Cl. A61K 39/395; A61K 35/12; A61K 31/53

(52) U.S. Cl. 424/173.1; 424/93.7; 424/529; 514/243

(57) ABSTRACT

An improved method for selectively depleting hypoxic cells within the bone marrow is disclosed. The method can be used to enhance engraftment of hematopoietic stem cells (HSCs) in the bone marrow of a host subject. Also disclosed is a method for treating a cancer within the bone marrow of a host subject.

### RELATED APPLICATIONS

This application claims priority to and the benefit of U.S. Provisional Patent Application No. 60/723,183, filed Oct. 3, 2005, for all subject matter common to said application. The disclosure of the above-mentioned application is hereby incorporated by reference herein in its entirety.

### GOVERNMENT FUNDING

This invention was made with government support under grant R01 10941-31 awarded by the NIH. The United States government has certain rights in the invention.

### BACKGROUND OF THE INVENTION

The hematopoietic system is maintained by a rare population of primitive hematopietic stem cells (HSCs) that are defined by the key feature of self-renewal, as well as the ability to generate multilineage progenitor populations that ultimately give rise to the functioning cells of blood and immune system. The normal mammalian hemaopoietic system is largely distributed around the adult body within the bone marrow and consists of quiescent stem cells and differentiated progenitors. The proliferative potential of HSCs is thus considerable as they have the unique ability to perpetuate themselves by self-renewal.

Functionally, HSCs are often defined in transplantation by their ability to engraft and maintain hematopoiesis in irradiated recipients (Weissman et al., 2001). Accordingly, it is important to effectively deplete or inactivate host HSCs in treating diseases involving HSCs, such as cancers, immune disorders, and transplant rejection. However, this has proven difficult, particularly because the frequency of HSCs is extremely low (estimated to be only 1 to 2 per so 100,000 bone marrow cells in competitive repopulation experiments (Harrison, 1980), making these cells more difficult to target and eradicate.

Current treatments typically involve administration of high doses of cytotoxic agents, usually in combination with radiation, which ablate not just HSCs, but all cells in the hematopoietic system. These therapies have clear drawbacks and severe toxic side effects. Accordingly, improved treatments for depleting HSCs, (e.g., prior to transplantation of donor HSCs to establish complete or mixed hematopoietic cell chimerism) would be beneficial.

### BRIEF SUMMARY OF THE INVENTION

The present invention provides a method for selectively depleting hypoxic cells, including HSCs, within the bone marrow by contacting the cells with a cytotoxic agent that specifically kills hypoxic cells, but not non-hypoxic cells, such that the hypoxic cells are selectively depleted. In one embodiment, the agent is administered to a subject in vivo prior to cellular or solid organ transplantation (e.g., bone marrow, donor mobilized peripheral blood, and umbilical cord blood) provided that the subject does not have a solid tumor. In such embodiments, the bone marrow can be irradiated or contacted with a chemotherapeutic agent prior or following administration of the agent that selectively kills hypoxic cells. In a particular embodiment, the HSCs are primitive HSCs, such as late forming Cobblestone Area-Forming Cells (CAFCs). Depletion of HSCs within the bone marrow can be measured, for example, in vitro in a CAFC assay or by long-term engraftment of congenically marked CD45.1 bone marrow transplanted in a subject.

In another aspect, the present invention provides a method for engrafting donor HSCs in the bone marrow of a host subject by administering to the subject an agent that selectively kills hypoxic cells, such that HSCs in the subject are depleted, and then administering HSCs from a donor subject. In a particular embodiment, the host subject is administered a chemotherapeutic agent or irradiation prior to or following administration of the agent that selectively kills hypoxic cells. In another particular embodiment, the host subject is administered a short-term immune modulating agent, such as a T-cell depleting antibody, in conjunction with (e.g., before, concurrently with, or following) administration of the agent that selectively kills hypoxic cells.

In another aspect, the present invention provides a method for treating a cancer within the bone marrow of a host subject by administering the subject an agent that selectively kills hypoxic cancer cells, such that the hypoxic cells in the subject are depleted, and then administering HSCs from a donor subject. In a particular embodiment, the cancer is a hematological cancer, such as a leukemia or a lymphoma. In another particular embodiment, the cancer is one which has metastasized to the bone marrow, such as neuroblastoma cells or breast carcinoma cells.

Preferred agents for use in the present invention selectively kill or deplete HSCs but not mature blood cells, so that the mature blood cells are maintained. In one embodiment, the agent is a bioreductive agent. In another embodiment, the agent is a hypoxia-activated prodrug. Particular agents which can be used in the invention include benzotriazines, such as Tirapazamine (TPZ; SR4233; 1,2,4-benzotriazin-3-amine 1,4-dioxide).

The invention also includes the use of other prodrugs that produce well-defined cytotoxins on reduction in hypoxic cells. These include nitroaromatic compounds (e.g. misonidazole; 1-methyl-3-(2-nitro-1-imidazolyl)-2-propanol and RB 6145; 2-nitroimidazole) (Adams et al. Int. J. Radiat. Oncol. Biol. Phys. 29, 231-238, 1994), anthraquinones (e.g. AQ4N; 1,4-Bis-[[2-(dimethylamino-N-oxide)ethyl]amino]5,8-dihydroxyanthracene-9,10-dione) (Patterson, L. R., Cancer Metastasis Rev. 12, 119-134, 1993; Patterson, L. H., Drug Metab. Rev. 34, 581-592, 2002; Patterson, L. H. et al. Br. J. Cancer 82, 1984-1990, 2000), the chloroquinoline DNA-targeting unit to 2-nitroimidazole (e.g. NLCQ-1; 4-[3-(2-Nitro-1-imidazolyl)-propylamino]-7-chloroquinoline hydrochloride) (Papadopoulou, M. V. et al. Clin. Cancer Res. 9, 5714-5720, 2003), dinitrobenzamide mustards, (e.g. SN 23862; 5-(N,N-bis(2-chloroethyl)amino)-2,4-dinitrobenzamide and SN 28343) (Siim, B. G., et al., Oncol. Res. 9, 357-369, 1997; Helsby, N. A. et al. Chem. Res. Toxicol. 16, 469-478, 2003), nitrobenzyl phosphoramidate mustards (Nitroheterocyclic Phosphoramidates) (Borch, R. F. et al., J. Med. Chem. 43, 2258-2265, 2000), nitrobeterocyclic methylquaternary salts (Nitroarylmethyl Quaternary Salts) (Tercel, M. et al. J. Med. Chem. 44, 3511-3522, 2001), cobalt(III) complexes (Wilson, W. R., et al., Int. J. Radiat. Oncol. Biol. Phys. 29, 323-327-1994) and indoloquinones (Everett, S. A., et al., Biochem. Pharmacol. 63, 1629-1639, 2002).

As hypoxia-inducible factor 1 alpha (HIF-1α) is a master regulator of the transcriptional response to low oxygen tensions, agents that have the ability to inactivate or deplete HSCs via inhibition of HIF-1α are included in the invention. Examples of HIF-1α inhibitors include camptothecin analogues (e.g., 1H-Pyrano(3′,4′:6,7)indolizino(1,2-b)quinoline-3,14(4H,12H)-dione, 4-ethyl-4-hydroxy-, (S)—) and topoisomerase (Topo)-I inhibitors (Rapisarda, A., et al., Cancer Res. 62, 4316-4324, 2002) and 3-(5′-hydroxymethyl-2′furyl)-1-benzyl indazole (YC-1) (Yeo, E. J. et al. J. Natl Cancer Inst. 95, 516-525, 2003). Another example of HIF-1α inhibitor include a drug called PX-478 (S-2-amino-3-[4′-N,N,-bis(2-chloroethyl)amino]phenyl propionic acid N-oxide dihydrochloride) which is about to enter the clinic (Garber K, J Natl Cancer Inst. 97, 1112-1114, 2005; Welsh, S et al., Molecular Cancer Therapeutics. 3, 233-244, 2004.)

The methods of invention can be used to treat and prevent a wide variety of diseases involving HSCs, and to enhance engraftment of donor stem cell transplants (e.g., to establish complete or mixed hematopoietic cell chimerism), with significantly less toxicity than current therapies in the treatment of malignant and non-malignant diseases, in the induction of immunological acceptance for cellular and/or solid organ transplantation (e.g., to induce a state of donor-specific immune tolerance), to prevent or reduce graft-versus-host disease (GvHD), to provide a platform for administering donor-leukocyte infusions (DLI), in the treatment of enzyme deficiency diseases, in the treatment of autoimmune diseases and in the transplant of genetically modified HSCs. Suitable combinations with short-term immune modulating agents (e.g., T cell-depleting antibodies) provide methods for engrafting hematopoietic stem cells from allogeneic and xenogeneic donors.

### BRIEF DESCRIPTION OF THE DRAWINGS

FIG. 1 shows the separation of different bone marrow fractions according to a Hoechst diffusion gradient. (A) Blue versus red fluorescence intensity after i.v. infusion of Hoechst dye (0.8 mg at 5 and 10 min.) with sorting gates for cell isolation. (B) CAFC frequencies with time in culture for the different fractions. (C) Early- and late-forming CAFC frequencies as a function of red Hoechst fluorescence.

FIG. 2 shows the long-term repopulation for donor cells (Ly5.1/CD45.1) sorted from high (R3) and low (R7) Hoechst perfused bone marrow. Bone marrow cell dose-responses are for myeloid (GR-1/CD11b) engraftment at 18 weeks post-BMT in 10 Gy irradiated recipients (Ly5.1/CD45.2). Similar results were obtained for T-cell (CD3) and B-cell (B220) chimerism. The number of mice for each cell dose group is indicated.

FIG. 3 shows pimonidazole metabolism at low oxygen tensions. This 2-nitroimidazole is non-toxic but forms adducts in hypoxic conditions that can be recognized by an antibody. This hypoxic marker has been used routinely to measure hypoxic cells in both clinical and experimental tumor specimens.

FIG. 4(A) shows low Hoechst perfusion and FIG. 4(B) shows positive staining with a hypoxic cell marker in the thymus.

FIG. 5 shows evidence for hypoxic Side Population (SP) cells in the bone marrow. Three different regions based on Hoechst dye efflux, non-SP(R1), high SP(R2) and low SP (R3) from mice injected 3 h previously with or without 120 mg/kg pimonidazole (n=5) were sorted and then intracellularly stained with a mouse anti-pimonidazole primary antibody and a goat anti-mouse IgG F(ab′)2 Alexa Fluor 488 secondary antibody. The sorted cells were also stained with a rat anti-CD45R PE-conjugated antibody to remove cross-reactivity of the goat antibody against B-cells.

FIG. 6 shows a model of oxygen diffusion in relation to the blood supply with location of the stem cell niche in a microenvironment of relatively low oxygen tension (hypoxic).

FIG. 7 shows the mechanism by which tirapazamine selectively kills hypoxic cells. Tirapazamine (TPZ) is a substrate for one-electron (1e-) reductases. The resulting free radical (TPZ•) undergoes spontaneous decay to an oxidizing hydroxyl radical (OH•) or an oxidizing benzotriazinyl radical (BTZ•). In the presence of oxygen, the TPZ radical is back-oxidized to the parent compound, producing a superoxide radical (O2-•) (This figure is reproduced from Brown & Wilson, 2004).

FIG. 8 is a depiction of CAFC content per hind limb expressed as percent of untreated (saline injected) control following 4×30 mg/kg tirapazamine (TPZ) 2×10 mg/kg Busulfex (BX) or 4 Gy gamma-irradiation. Data represents the mean of bone marrow cell samples pooled from 3-4 individual mice with 95% confidence limits.

FIG. 9 is a conceptual illustration of preferential depletion of hypoxic normal HSCs by TPZ and the subsequent engraftment and repopulation of transplanted donor HSCs.

FIG. 10 is a conceptual illustration of preferential depletion of malignant HSCs that are hypoxic and therefore susceptible to TPZ treatment, as well as the subsequent eradication of the disease.

FIG. 11 shows that 5-FU selectively depletes early-forming CAFCs. 5-FU was administered i.p. to B6 recipients at a single dose of 150 mg/kg. At 2 days, the femoral and tibial bone marrow was harvested, pooled from mice and plated for estimate of CAFC content per hind limb. Error bars represent 95% confidence intervals.

FIG. 12 shows increased Hoechst perfusion in bone marrow after 5-FU. At different times after 5-FU, Hoechst dye was injected i.v. (0.8 mg at 5 and 10 min) and the bone marrow analyzed for intensity of Hoechst uptake. The plotted graph gives the red fluorescence intensity for cells at the lower 1% level to show how the Hoechst gradient is shortened at 1 to 6 days after 5-FU and provides evidence for improved oxygenation of the cells that remain after treatment.

FIG. 13 shows that the number of SP cells in bone marrow is decreased after 5-FU. At different times after 5-FU, bone marrow was harvested, nucleated cells counted and incubated in Hoechst dye for subsequent analysis on efficient (lower gate) and poor (higher gate) dye effluxing cells in the SP tail. While the percent of SP cells appeared to increase from day 1 to day 4, the cell yield continued to decrease and thus the actual number of SP cells per hind limb remained low.

FIG. 14 shows a conceptual illustration of reoxygenation of HSCs after 5-FU treatment. This model provides an explanation for both a decrease in the Hoechst diffusion gradient concomitant with increased oxygenation of HSCs and a loss of the SP phenotype as the latter is determined by ABCG2 expression that is controlled by HIF-1α (Krishuiamurthy et al. 2004).

### DETAILED DESCRIPTION OF THE INVENTION

The present invention relates generally to an improved method for removing normal or malignant hematopoietic stem cells (HSCs) in bone marrow, including HSCs and metastasized cancer cells. The present invention also provides an improved method of treating a cancer within the bone marrow of a host subject. Existing methods for depleting HSCs and hypoxic cancer cells within the bone marrow prior to transplantation of donor HSCs are non-selective and thus significantly deplete other cells within the bone marrow, thereby having considerable toxic side effects. However, as part of the present invention it was discovered that HSCs, as well as other cells, such as cancer cells which have metastasized to the bone marrow, can be selectively depleted by virtue of the fact that they reside within a hypoxic niche in the bone marrow. Accordingly, in a particular embodiment, the methods of the present invention selectively target and deplete hypoxic cells within the bone marrow using agents that are active only under hypoxic conditions, thereby reducing or eliminating the undesirable side effects associated with existing therapies. The present invention is particularly useful for the treatment of a variety of non-solid malignancies, such as hematological malignancies and cancers which have metastasized to the bone marrow. In a particular embodiment, the method of the invention is used to selectively remove hypoxic cells in the bone marrow of a subject, who does not have a solid tumor (i.e., to treat only hematological (non-solid) malignancies.)

In order that the present invention may be more readily understood, the following terms are defined as follows:

The term “depleting” refers to inactivating, killing or reducing the number of HSCs.

The term “selectively” refers to the ability of the agent to target hypoxic cells (e.g., HSCs and/or cancer cells) without targeting cells that reside in a non-hypoxic environment (e.g., non-HSC progenitors, mature blood cells).

The term “hematopoietic stem cells” (HSCs) refers to pluripotent cells which have an extensive capacity for self-maintenance and self-renewal and are capable of differentiating into a variety of progenitor cell types. This term includes primitive HSCs, e.g., late forming CAFCs.

The term “self-renewal” refers to the fact that these cells can give rise to progeny identical in appearance and differentiation potential.

The term “cancer” refers to any malignant growth caused by abnormal and uncontrolled cell division, provided that the malignant growth is not a solid tumor and provided that the host subject does not have a solid tumor. This term includes, but is not limited to, hematological cancers (e.g., leukemias and lymphomas) and cancers which have metastasized to the bone marrow (e.g., neuroblastoma cells and breast carcinoma cells).

The term “CAFC” refers to Cobblestone Area Forming Cells, which are immature HSCs. These cells include “early forming” and “late forming” CAFCs. “Late Forming CAFCs” are typically immature HSCs which appear in culture at day 25 or more (e.g., 28-35 days) and reside in the stromal layer. These cells form colonies known as “Cobblestone Areas.”

The term “hypoxic cells” refers to cells which reside in a low oxygenated environment, e.g., an oxygen tension of less than pO₂ of 10 mm Hg. The term “hypoxia-activated prodrug” refers to a drug that is initially inactive, but becomes activated in a hypoxic or low oxygenated environment.

The term “complete chimerism” or “complete hematopoietic stem cell chimerism” refers to the successful engraftment of donor HSCs in the host subject, wherein the donor HSCs and ascendant populations in, e.g., the blood, constitute more than 99% in the host.

The term “mixed chimerism” or “mixed hematopoietic stem cell chimerism” refers to a state of varying proportions of engrafted donor HSCs and resident host HSCs in the transplant recipient, wherein the donor HSCs and ascendant populations in, e.g., the blood, constitute levels of between 1 and 99%.

As stated above, the methods of the present invention provide improved methods for selectively depleting hypoxic cells (e.g., HSCs and/or cancer cells) within the bone marrow, without substantially depleting mature blood cells, which are less toxic than existing myeloablative procedures. The HSCs which are selectively depleted may be any HSCs within the bone marrow, including primitive HSCs, such as late forming Cobblestone Area-Forming Cells (CAFCs).

In one embodiment of the invention, the method involves selectively depleting hypoxic cells (e.g., HSCs and/or cancer cells) in a host subject followed by engraftment of donor HSCs in the subject. The donor HSCs may be derived from any suitable source, including donor bone marrow, donor peripheral blood cells (e.g., cytokine mobilized peripheral blood cells) and donor umbilical cord blood and may be obtained using any suitable means known in the art. For example, to obtain donor HSCs from cytokine mobilized peripheral blood cells a cytokine (e.g., G-CSF; Granulocyte Colony Stimulating Factor) can be administered to a donor, which causes the HSCs to migrate from the bone marrow to the peripheral blood where the cells can then be collected before they are administered to a host recipient Furthermore, the donor cells may be obtained from any suitable donor, including an allogeneic donor or xenogeneic donor. The so donor HSCs may also be genetically modified HSCs.

The methods of the present invention are designed to selectively deplete hypoxic cells (e.g., HSCs and/or cancer cells) within the bone marrow by administering an agent that is toxic to hypoxic cells (e.g., cells that exist at a low oxygen tension of less than about pO₂ of 10 mm Hg). A variety of such agents are well known in the art. For example, the agent may be a bioreductive agent or a hypoxia-activated prodrug which becomes active in a low oxygen environment. Such agents include, but are not limited to, benzotriazines and benzotriazine-related compounds. In one embodiment, the agent is Tirapazamine (TPZ) (1,2,4-benzotriazin-3-amine 1,4-dioxide) or an analog or derivative of Tirapazime. Tirapazamine and other benzotriazine compounds are well known in the art and can be prepared and administered, as described, for example, in U.S. Pat. No. 3,957,779, U.S. Pat. No. 5,175,287, U.S. Pat. No. 5,672,702,U.S. Pat. No. 6,121,263, U.S. Pat. No. 6,319,923, U.S. Pat. No. 6,063,780, U.S. Pat. No. 6,277,835, and WO 97/20828, the contents of which are incorporated herein by reference.

Other prodrugs that produce well-defined cytotoxins on reduction in hypoxic cells include nitroaromatic compounds (e.g. misonidazole; 1-methyl-3-(2-nitro-1-imidazolyl)-2-propanol and RB 6145; 2-nitroimidazole) (Adams, G. E. et al., Int. J. Radiat. Oncol. Biol. Phys. 29, 231-238, 1994), anthraquinones (e.g. AQ4N; 1,4-Bis-[[2-(dimethylamino-N-oxide)ethyl]amino]5,8-dilhydroxyanthracene-9,10-dione) (Patterson, L. H., Cancer Metastasis Rev. 12, 119-134, 1993; Patterson, L. H., Drug Metab. Rev. 34, 581-592, 2002; Patterson, L. H. et al., Br. J. Cancer 82, 1984-1990, 2000), the chloroquinoline DNA-targeting unit to 2-nitroimidazole (e.g. NLCQ-1; 4-[3-(2-Nitro-1-imidazolyl)-propylamino]-7-chloroquinoline hydrochloride) (Papadopoulou, M. V. et al., Clin. Cancer Res. 9, 5714-5720, 2003), dinitrobenzamide mustards, (e.g. SN 23862; 5-(N,N-bis(2-chloroethyl)amino)-2,4-dinitrobenzamide and SN 28343) (Siim, B. G., et al., Oncol. Res. 9, 357-369, 1997; Helsby, N. A. et al., Chem. Res. Toxicol. 16, 469-478, 2003), nitrobenzyl phosphoramidate mustards (Nitroheterocyclic Phosphoramidates) (Borch, R. F. et al., J. Med. Chem. 43, 2258-2265, 2000), nitroheterocyclic methylquaternary salts (Nitroarylmethyl Quaternary Salts) (Tercel, M. et al., J. Med. Chem. 44, 3511-3522, 2001), cobalt(III) complexes (Wilson, W. R., et al., Int. J. Radiat. Oncol. Biol. Phys. 29, 323-327, 1994) and indoloquinones (Everett, S. A. et al., Biochem. Pharmacol. 63, 1629-1639, 2002).

In another embodiment, agents that have the ability to inactivate or deplete HSCs via inhibition of HIF-1α are included in the invention. Examples of HIF-1α inhibitors include camptothecin analogues (e.g., 1H-Pyrano(3′,4′:6,7)indolizino(1,2-b)quinoline-3,14(4H,12H)-dione, 4-ethyl-4-hydroxy-, (S)—) and topoisomerase (Topo)-I inhibitors (Rapisarda, A. et al., Cancer Res. 62, 4316-4324, 2002) and 3-(5′-hydroxymethyl-2′furyl)-t-benzyl indazole (YC-1) (Yeo, E. J. et al., J. Natl Cancer Inst. 95, 516-525, 2003). Another example of HIF-1α inhibitor include a drug called PX-478 (S-2-amino-3-[4′-N,N,-bis(2-chloroethyl)amino]phenyl propionic acid N-oxide dihydrochloride) which is about to enter the clinic (Garber K, J Natl Cancer Inst. 97, 1112-1114, 2005; Welsh, S et al., Molecular Cancer Therapeutics. 3, 233-244, 2004.)

The hypoxia-activated agent of the present invention may be administered via any suitable route of administration. As will be appreciated by the skilled artisan, the best route for in vivo administration may vary depending upon the patient or desired result. Suitable routes of administration for agents of the invention include, but are not limited to, intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Hypoxia-activated agents of the invention are preferably administered to a subject in a suitable pharmacological form (e.g., as a pharmaceutical composition). For example, the agent can be formulated with carriers and other pharmaceutically acceptable compounds, that will protect the agent against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Such formulations are well known to those skilled in the art. See, e.g., Sustained and Controlled Release Drug Delivery Systems, J. R. Robinson, ed., Marcel Dekker, Inc., New York, 1978.

For parenteral administration, it is especially advantageous to formulate the agent in dosage unit form for ease of administration and uniformity of dosage. Dosage unit form as used herein refers to physically discrete units suited as unitary dosages for the particular individual to be treated; each unit containing a predetermined quantity of active compound calculated to so produce the desired therapeutic effect in association with the required pharmaceutical carrier.

When administered parenterally, an agent of the invention will normally be formulated in a unit dosage injectable form (solution, suspension, emulsion) with a pharmaceutically acceptable vehicle. Such vehicles are typically nontoxic and non-therapeutic. Examples of such vehicles are water, aqueous vehicles such as saline, Ringer's solution, dextrose solution, and Hank's solution and non-aqueous vehicles such as fixed oils (e.g., corn, cottonseed, peanut and sesame), ethyl oleate, and isopropyl myristate. The vehicle may contain minor amounts of additives such as substances that enhance solubility, isotonicity, and chemical stability, e.g., antioxidants, buffers, and preservatives.

Hypoxia-activated agents of the present invention are administered to a subject in an amount and for a sufficient time period to achieve selective depletion of hypoxic cells (e.g., HSCs or cancer cells) in bone marrow. The appropriate dosage of the agent will depend on factors such as the disease state, severity of the condition to be alleviated, age, sex, and weight of the individual. Adjustment of dosage regimens for known chemotherapeutics is well within the routine skill of the art. It is to be further understood that for any particular subject, specific dosage regimens should be adjusted over time according to the individual need and the professional judgment of the person administering or supervising the administration of the compositions. The agent may be administered once, or may be divided into a number of smaller doses to be administered at varying intervals of time.

Hypoxia-activated agents of the present invention can be administered alone or in combination with one or more other therapeutic or pharmaceutical agents (e.g., prior to or following the administration of donor bone marrow, donor cytokine mobilized peripheral blood, or donor umbilical cord blood). For example, in one embodiment of the invention, the agent may be combined with a short-term immune modulating agent, such as a T cell-depleting antibody, which functions to deplete or inactivate immune cells in the host.

Hypoxia-activated agents of the present invention can also be administered in combination with one or more myeloablative therapies, such as radiation therapy or chemotherapy. The agents may also be administered in combination with one or more chemotherapeutic agents. Such adjunctive therapies may be administered prior to, subsequent to, or in conjunction with administration of the hypoxia-activated agent. Particular chemotherapeutic agents include, but are not limited to, All-trans retinoic acid, Aminoglutethimide, Azacitidine, Azathioprine, Bleomycin (Blenoxane), Busulfan (Myeleran), Carboplatin, Carboplatinuin (Paraplatin), Carmustine (BCNU), Capecitabine, CCNU (Lomustine), Chlorambucil (Leukeran), 2-Cholrodeoxyadenosine (2-CDA; Cladribine, Leustatin), Cis-platinum (Platinol), Cisplatin (cis-DDP), Cisplatin bleomycin sulfate, Chlorambucil, Cyclophosphamide (Cytoxanl CTX), Cyclophosphamide hydroxyurea, Cytarabine (Ara-C; cytosine arabinoside), Daunorubicin (Cerubidine), Dacarbazine (DTIC; dimethyltriazenoimidazolecarboxamide), Dactinomycin (actinomycin D), Daunorubicin (daunomycin; rubidomycin), Diethylstilbestrol, Docetaxel (Taxotere), Doxifluridine, Doxolubicin (Adriamycin), Epirubicin, Ethinyl estradoil, Etopaside (VP-16, VePesid), Fluorouracil (5-Fu; Floxuridine, fluorodeoxyuridine; FUdR), Fludarabine (Fludara), Flutamide, Fluoxymesterone, Gemcitabine (Gemzar), Herceptin (Trastuzumab; anti-HER 2 monoclonal antibody), Hydroxyurea (Hydrea), Hydroxyprogesterone caproate, Idarubicin, Ifosfamide (Ifex), Interferon alpha, Irinotecan (CPT-11), L-Asparaginase, Leuoprolide, Mechlorethamine, Medroxyprogesterone acetate, Megestrol acetate, Melphelan (Allceran), Mercaptopurine (6-mercaptopurine; 6-MP), Methotrexate (MTX; arnethopterin), Mitomycin (mitomycin C), Mitotane (o,p′-DDD), Mitoxantrone (Novantrone), Oxaliplatin, Paclitaxel (Taxol), Pemetrexed, Pentostatin (2-deoxycoformycin), Plicamycin (mithramycin), Prednisone, Procarbazine (Matulane; N-methylhydrazine, MIH), Rituxin (Rituximap), Semustine (Methyl-CCNU), Streptozocin, Tamoxifen, Teniposide, Tertiposide, Testosterone propionate, Thioguanine (6-thioguanine; TG), Thiotepa, Tomudex (Raltitrexed), Topotecan (Hycamtin; (S)-10-[(dimethylamino) methyl]-4-ethyl-4,9-dihydroxy-1H-pyrano[3′, 4′), Treosulfan (Ovastat), Valrubicin, Vinblastine (VLB; Velban), Vincristine (Oncovin), Vindesine, and Vinorelbine Navelbine).

Selective depletion of hypoxic cancer cells land HSCs can be tested by any means known in the art. In one embodiment, depletion of HSCs is measured in vitro using the Cobblestone Area-Forming Cells (CAFC) assay, an assay which is well known in the art (see e.g., Neben et al, 1993; Ploemacher et al., 1991 and Ploemacher et al, 1989). Alternatively, depletion of HSCs can be measured in vivo by long-term engraftment of congenically marked CD45.1 bone marrow transplanted in a subject. Long-term engraftment is defined by stable donor-type chimerism at and beyond a period of 16 weeks after bone marrow transplant according to the percent of peripheral leukocytes bearing the donor-specific marker. In all cases, selective depletion should achieve the desired clinical effect. When administering bone marrow in vivo, it is desirable to deplete 30 to 100% of host HSCs, to promote partial or complete engraftment of donor HSCs. For example, it is desirable to deplete 30% or greater of host HSCs, more preferably 50% or greater of host HSCs, and most preferably 75% or greater of host HSCs. When administering in vivo to treat immune or genetic diseases, depletion can be measured by prevention or a reduction in the symptoms of the disease.

In certain embodiments, the methods and compositions of the present invention can be used to enhance engraftment of donor stem cell transplants (e.g., to establish complete or mixed hematopoietic cell chimerism), with significantly less toxicity than current therapies in the treatment of malignant and non-malignant diseases, in the induction of immunological acceptance for cellular and/or solid organ transplantation (e.g., to induce a state of donor-specific immune tolerance), to prevent or reduce graft-versus-host disease (GvHD) and to provide a platform for administering donor-leukocyte infusions (DLI),

In addition to enhancing engraftment of donor stem cell transplants, the methods and compositions of the present invention can be used to treat and prevent a wide variety of malignant and non-malignant diseases. Such diseases include autoimmune diseases, enzyme deficiency diseases and non-solid cancers. Specifically, the cancers may, include, but are not limited to cancers which have metastasized to the bone marrow (e.g., neuroblastoma cells and breast carcinoma cells) and hematological cancers (e.g., leukemias, lymphomas, multiple myelomas, myeloproliferative disorders and myelodysplastic syndromes).

The methods of the present invention may also be used to treat or prevent a wide variety of diseases through the administration of genetically modified donor HSCs to HSC depleted host bone marrow. In particular, the methods of the present invention can be used to enhance or facilitate engraftment of donor stem cell transplants (e.g., to establish complete or mixed hematopoietic cell chimerism) and to treat or prevent transplant rejection (e.g., cell, tissue or organ transplants).

The present invention is further illustrated by the following examples which should not be construed as further limiting. The contents of all figures and all references, patents and published patent applications cited throughout this application are expressly incorporated herein by reference. Those skilled in the art will recognize, or be able to ascertain using no more than routine experimentation, many equivalents to the specific embodiments of the invention described herein. Such equivalents are intended to be encompassed by the following Examples and claims.

### EXAMPLES

The following examples demonstrate that HSCs (e.g., primitive HSCs known as late Cobblestone Area Forming Cells (CAFCs)) reside in a defined hypoxic niche within bone marrow and can be selectively targeted using hypoxia-activated cytotoxins to treat a variety of disorders. About a 100-fold difference in frequency of the primitive CAFC day-35 subset was observed across a Hoechst perfusion gradient (Example 1), which is considerably higher than the 2- to 4-fold differences noted in the other approaches aimed at defining the location of the bone marrow stem cell niche (Lord, 1990; Nilsson et al., 2001). The relative change in CAFC day-35 frequencies among the different sorted fractions was reflected in the ability of these same populations to repopulate long-term irradiated transplant recipients to further validate the CAFC assay as a reliable means of quantifying HSCs in mouse bone marrow. The evidence supporting the hypoxic nature of HSCs (Example 2) is consistent with the notion that the distribution of Hoechst fluorescence after intravenous delivery of the dye simulates an oxygen gradient. Alterations in this gradient following 5-FU treatment (Example 5) confirms that alterations in oxygen levels play a fundamental role in controlling HSC homeostasis.

### Materials and Methods

Mice: C57BL/6J male mice (with Ly 5.2 marker) obtained from Jackson Laboratories were used for most of the experiments proposed. Mice were maintained in a specific pathogen-free microisolator environment. For some in vivo repopulation assays, B6.5JL-Ptprc^{a} Pep3^{b}/BoyJ congenic mice (with Ly5.1 marker) obtained from Jackson Laboratories were used as donors.
Bone marrow, thymus, harvest: Bone marrow cells were harvested from mice by crushing the tibias and femurs from the hind limbs in HBSS containing 2% FBS and 10 Mm mM HEPES buffer (HBSS+). Bone marrow cells, thymocytes and splenocytes were passed through 21 gauge needles to get single cell suspensions. The cellularity of bone marrow and thymus was measured by counting total live cells (using trypan blue) and total WBCs (using crystal violet in 3% acetic acid) on a hemocytometer.
Measurement of Hoechst perfusion in vivo in mice: The Hoechst dye perfusion in mice was done according to methods previously described in the literature (Durand et al., 1990; Olive et al., 2000; Olive et al., 2002). Briefly, C57BL/6J mice were injected i.v. via the retro-orbital sinus under isoflurane anesthesia with two doses (0.8 mg/mouse) of Hoechst dye (Sigma) at 10 and 5 min before bone marrow harvesting, a period determined to be insufficient for active dye exclusion in vitro. Tibias and femurs were placed immediately on ice, crushed in a pre-cooled mortar and pestle, filtered and suspended in cold HBSS+. Thymus was harvested and thymocyte cell suspension made by passing the cells through 21 gauge needle. The dual emission wavelength of Hoechst fluorescence in bone marrow and thymus was assessed on a logarithmic scale using flow cytometry with exclusion of propidium iodide positive cells.
Pimonidazole binding: The detection of hypoxic cells in bone marrow and thymus was done by pimonidazole binding using methods described by Olive et al. (Olive et al., 2000; Olive et al., 2002). Briefly, the mice were injected i.p. with pimonudazole hydrochloride (Chemicon International) at 120 mg/kg dose and the bone marrow as well as thymus were harvested 3 hrs post-injection. The cell population was fixed and permeabilized using a kit from Chemicon and stained with mouse monoclonal anti-pimonidazole antibody (Hypoxyprobe™, Chemicon) and a goat anti-mouse IgG F(ab′)2 Alexafluor 488 secondary antibody. In some experiments, mouse monoclonal FITC-labelled anti-pimonidazole antibody were used to detect pimonidazole binding to the cells. The fluorescent intensity for staining was measured using flow cytometry.
Isolation of SP cells: The bone marrow cells were stained with Hoechst 33342 (Sigma) as described by Mulligan and colleagues (Goodell et al., 1996). In brief, the cells were centrifuged, pelleted, and resuspended at 10⁶ cells per ml in DMEM+. Hoechst 33342 was added at a final concentration of 5 μg/ml. As a negative control, 50 μM of Verapamil was added to a small aliquot. All the cells were then incubated for 90 min at 37° C., then pelleted by centrifugation and resuspended in cold HBSS+. The cells were then used for antibody staining (if further selection was needed) or for cell sorting. Before flow cytometric and sorting analysis, the samples were stained with 2 μg/ml Propidium Iodide (PI) to identify nonviable cells. Hoechst effluxing SP cells were detected by flow cytometric analysis on a dual-laser Mo-Flo (Cytomation, Inc., Ft Collins, Colo., USA) as described previously (Goodell et al., 1996).
Floss Cytometry: Flow cytometric analysis and sorting was performed on a dual-laser Mo-Flo (Cytomation, Inc.). The Hoechst dye was excited with U.V. excitation and its fluorescence emission was measured at two wavelengths using a 450/65 BP (450/65 nm band pass filter) and a 630/30 BP (630/30 nm band pass filter) optical filters (Omega Optical Inc.). A 510 DCLP (510 nm long pass dichroic mirror) was used to separate the emission wavelengths. Propidium Iodide (PI) fluorescence was also measured through the 630 BP (having been excited with U.V. excitation). Hoechst “blue” utilizes the 450 BP filter, the standard analysis wavelength for Hoechst 33342 DNA content analysis. Dead and dying cells positive for PI are seen on the far right of the Hoechst “red” (630 BP) axis and excluded. Fluorescence from the Hoechst dye was acquired on linear scales for SP cells and on log scale for Hoechst perfusion gradients. The gating on forward and side scatter were not stringent: only erythrocytes and debris were excluded.
Cobblestone Area Forming Cell (CAFC) Assay: The murine stromal cell line FBMD-1 cells (obtained from Dr. Rob Ploemacher, Erasmus University, Rotterdam, The Netherlands) was plated on 0.3% porcine gelatin (Sigma Chemical Co., St. Louis, Mo., cat. # G-2500) coated 96 flat well plates in IMDM (Life Technologies Gibco BRL Products, cat. # 31980-030), 10⁻⁶M hydrocortisone (Sigma Chemical Co., cat #H-0135), 10⁻⁴ M beta-mercaptoethanol (ICN Biochemicals Inc., cat, # 194705), 10% fetal bovine serum (Life Technologies Gibco BRL Products, cat. #10437-828), 100 units/ml of penicillin and 100 μg/ml of streptomycin (Biowhittaker cat #17-502E) and incubated at 37° C. in 5% CO₂. After the stromal cells reach confluency, the harvested BMCs were pooled for each experimental group and plated in Iscove's modified Dubecco's medium (IMDM; Life Technologies Gibco BRL Products, cat. # 31980-030), 10⁻⁶ M hydrocortisone (Sigma Chemical Co., cat #H-2270), 10⁻⁴ M beta-mercaptoethanol (Sigma, cat, # M-7522), 20% horse serum (Biowhittaker, cat. #14-403F), 100 units/ml of penicillin and 100 ug/ml of streptomycin (Biowhittaker cat # 17-502E) and incubated at 33° C. in 5% CO². Six to nine dilutions per experimental group with 20 wells per dilution were used. Positive wells containing at least one cobblestone cells were counted at 7 to 35 days after overlay under a Nikon Diaphot-TMD inverted microscope. The number of CAFCs per femur and the 95% confidence intervals were calculated using LCALC computer program (Stem Cell Technologies) or LDA computer program according to the method devised by Fazekas de St. Groth (1982).
Long term repopulation in vivo: Long term repopulation assay (LTRA) in vivo is a standard primitive stein cell assay which is well known in the art. The LTRA measures the long-term repopulating ability of a test stem cell population in vivo (Harrison, 1980). This assay can be used to measure or confirm selective-depletion of HSCs in vivo. The repopulation assays was performed by using the congenic Ly5.1/Ly5.2 system as described (Spangrude and Scollay, 1990). For determination of LTRA, varying numbers of bone marrow test cells (1×10⁻¹×10) from B6 (Ly5.1) mice were mixed with a 2×10⁵ of nonSP supporting bone marrow cells harvested from recipient B6-Ly5.2 mice. The mixtures were injected into groups of 5-10 lethally irradiated B6Ly5.2 recipients (1000 cGy dose TBI). Absence of endogenous marrow repopulation was determined by injecting one group Ly5.2 with control cells only. Recipients were bled at selected time points (3-6 months) and the donor cell repopulationg ability cells were determined by staining leukocytes with Ly5.1 antibodies. In addition, the percentage of donor-derived T cells, B cells, and myeloid cells was determined by co-staining with anti-CD3, anti-B220, anti-Gr-1 and anti-Mac-1 antibodies.
Statistics: For statistical analysis of CAFC assays, the Poisson-based LDA calculation was used for CAFC frequencies and 95% confidence limits (CL) were calculated for each subset. When the data available was from only a single experiment, non-overlapping 95% CL was interpreted as a significant result (p<0.05).

### Example 1

### Evidence That Different Hematopoietic Subsets Are Distributed Along a Hoechst Dye Perfusion Gradient That May Reflect the Distance from Blood Vessels and Level of Oxygenation

A number of previous animal studies have used the intravenous injection of the diffusible dye Hoechst 33342 to visualize tissue sections under fluorescence microscopy the perfusion of solid tumors in relation to hypoxia. Apart from situations in which tumor blood perfusion fluctuates leading to “acute” or “transient” hypoxia (Brown, 1979), most reports describe the location of hypoxic cells at a relatively constant from blood vessels (Bernsen et al., 2000; Chaplin et al., 1987; Durand et al., 1990; van Laarhoven et al., 2004) to indicate chronic or so diffusion-limited hypoxia, according to the classical model of Thomlinson and Gray (Thomlinson and Gray, 1955): Peggy Olive and colleagues have described an elegant series of experiments in which the Hoechst dye diffusion gradient can be more quantifiably characterized using flow cytometry following disaggregation of tumor tissue into a cell suspension and provided evidence on how the intensity of Hoechst staining simulated the degree of oxygenation (Olive et al., 2000).

In the present study, a similar approach was used to evaluate in vivo Hoechst uptake in bone marrow cells. These results were compared with perfusion of thymus, a tissue that has recently been shown to be grossly hypoxic according to hypoxic marker and oxygen electrode measurements (Hale et al., 2002).

C57BL/6J mice were intravenously injected with two doses (0.8 mg/mouse) of Hoechst dye at 10 and 5 min before bone marrow harvesting, a period that determined to be insufficient for active-dye exclusion in vitro. Tibias and femurs were placed immediately on ice, crushed in a pre-cooled mortar and pestle, filtered and suspended in cold HBSS+ and the dual emission wavelength of Hoechst fluorescence was assessed on a logarithmic scale with exclusion of propidium iodide positive cells. Analysis was also performed on harvested thymocytes from the same mice.

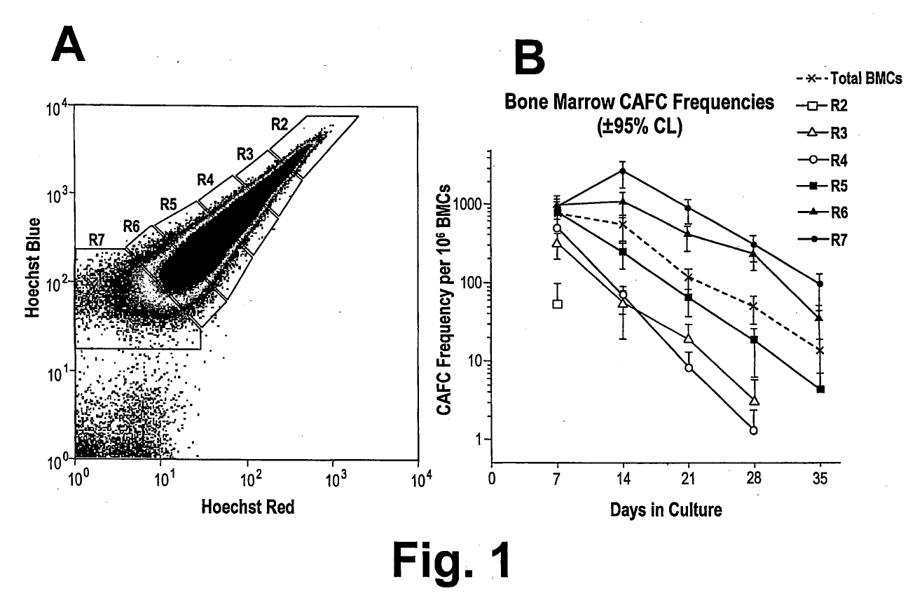

FIG. 1A shows a wide distribution of Hoechst staining for bone marrow covering 3 logs of fluorescence intensity. Cells were then isolated on the FACS machine based on six different gated regions with decreasing Hoechst fluorescence (R2 to R7) and their proliferative potential in vitro was assessed by plating the sorted cells on confluent cultures of the bone marrow stromal cells line (FBMD-1) in 96-well plates over a series of limiting dilutions according to Ploemacher and colleagues (Ploemacher et al., 1991; Ploemacher et al., 1989). Cobblestone area forming cell (CAFC) frequencies were determined at weekly intervals that have been well-established from numerous past studies to reflect a spectrum of hematopoietic subsets. Specifically, day 7 and 14 CAFCs correspond to early progenitor cells and to CFU-spleen-day 12 cells while the later-forming day 28 and 35 CAFCs correlate with HSCs capable of long-term repopulation in transplanted recipients (Down et al., 1994; Down et al., 1995; Down and Ploemacher, 1993; Ploemacher et al., 2004; Ploemacher et al., 1991; Westerhof et al., 2000).

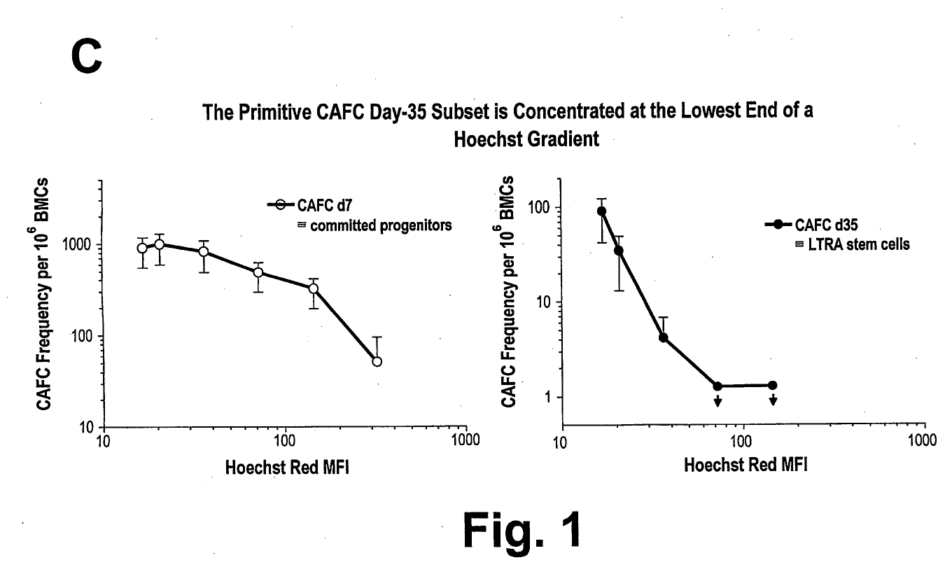

As shown in FIGS. 1B and C, the primitive CAFC subset appearing at day 28 to 35 in culture was shown to be progressively enriched with decreasing Hoechst fluorescence while the day 7 CAFC subset frequencies remained relatively constant. In the highest Hoechst-stained cells (R2 region), there was an overall deficit of CAFCs, presumably because much of this fraction consists of circulating blood. Sorted cells from the far ends of this gradient were also analyzed in a competitive it vivo repopulation assay. In this case the isolated cells from Ly5.1 congenic mice were injected i.v. into lethally irradiated (10 Gy) recipient Ly5.2 recipients at different cell concentrations together with 105 short-term repopulating non-SP recipient-type supporting cells to ameliorate the acute toxic effects of irradiation. Assessment of donor-type blood cell chimerism at 18 weeks post-BMT showed results that were in accordance with the primitive AFC frequency results: cells with the lowest Hoechst fluorescence (R7 region in the gradient) exhibited 10 times more repopulating ability than total unfractionated bone marrow cells. In contrast, cells having high fluorescence (R3 region in the gradient) showed low level of engraftment (FIG. 2).

### Example 2

### Side Population (SP) Bone Marrow Cells Are Positive For a Hypoxic Cell Marker

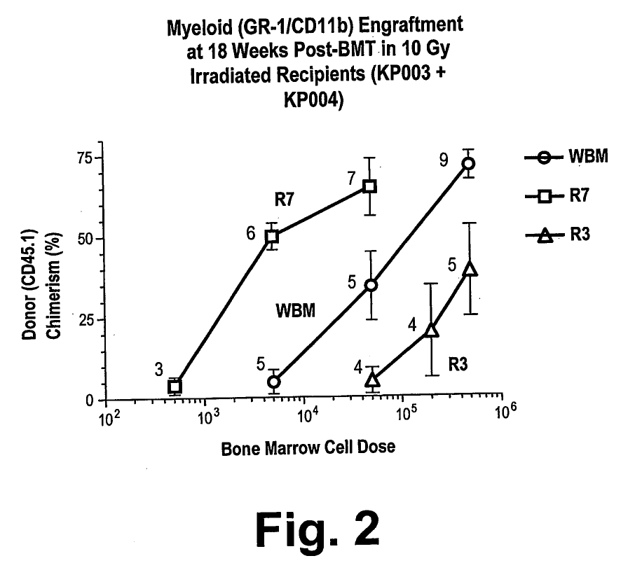

In the present study, reductive 2-nitroimidazole compound pimonidazole was utilized, which, when administered in vivo, forms adducts in hypoxic regions (less than pO₂ of 10 mm Hg) that can then be identified by anti-pimonidazole antibodies (FIG. 3).

To detect hypoxic cells in bone marrow and thymus, 120 mg/kg pimonidazole was administered i.p. to mice and bone marrow and thymocytes were harvested after 3 hrs post-injection. Bone marrow cells were stained with Hoechst 33342 in vitro and SP cells effluxing the dye (along with non-SP cells) were isolated by FACS. Thymocytes and sorted bone marrow populations were then fixed, permeabilized and incubated with the primary mouse anti-pimonidazole antibody (HypoxyProbe, Chemicon) and a goat anti-mouse secondary for staining and detection by flow cytometry.

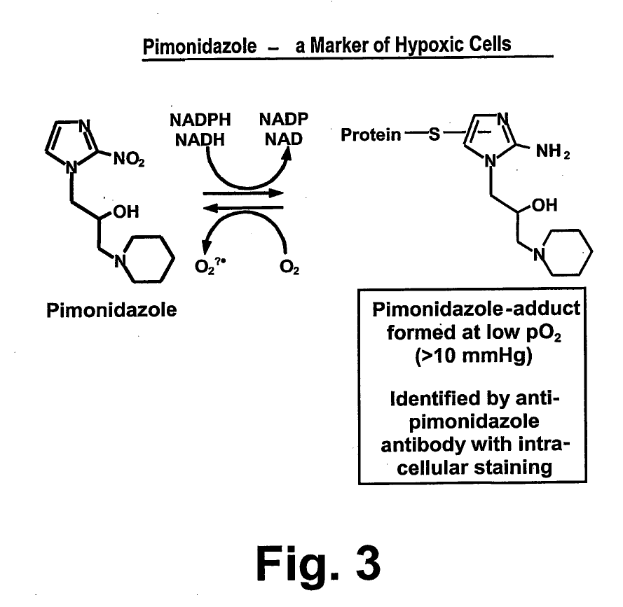

The thymus was found to be very poorly perfused after Hoechst injection and, as expected, the majority of thymocytes were positive for the hypoxic marker (pimonidazole adducts) confirming the previous report by Hale et al (2002) that thymocytes are hypoxic in vivo (FIG. 4).

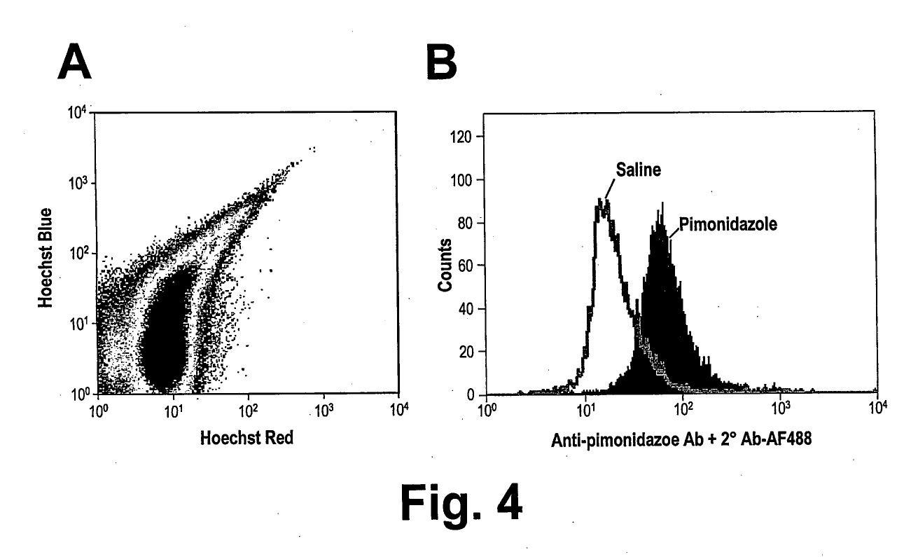

In the bone marrow fractions, non-SP cells had only a small shift of pimonidazole staining compared to staining on SP cells. Within SP cells, the low dye effluxing fraction showed increased anti-pimonidazole staining, and high dye effluxing SP cells had the highest so pimonidazole staining (FIG. 5). As discussed earlier, the high dye effluxing SP cells (“tip” SP cells) are the most primitive stem cells in mouse bone marrow.

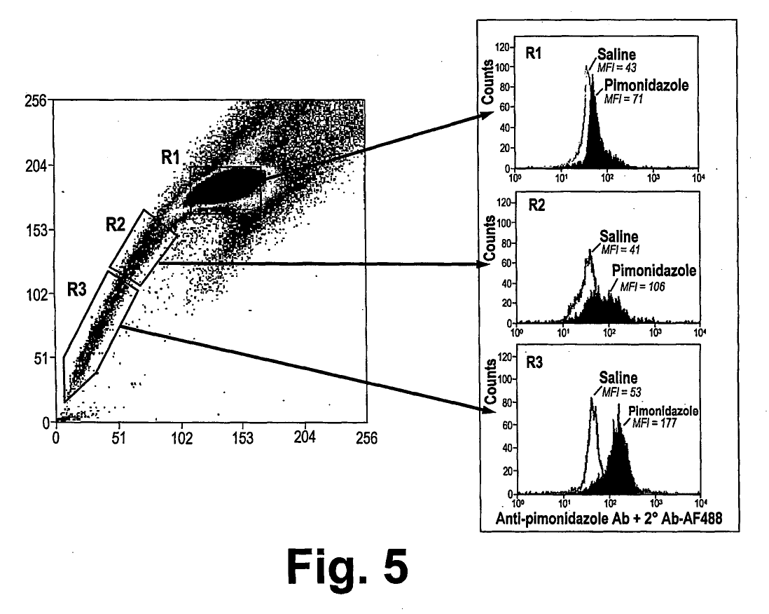

Collectively, these data represent the first direct evidence that the most primitive hematopoietic cells in the mouse bone marrow are relatively hypoxic and are consistent with their location at the lowest end of an oxygen gradient. Thus, the Hoechst staining appears to simulate oxygen diffusion and spatially defines HSCs as being the furthest from the blood supply and in the endosteal region as illustrated in FIG. 6.

### Example 3

### Selective Depletion of Late-Forming Cobblestone Area Forming Cell (CAFC) Subsets in Bone Marrow by Tirapazamine Treatment In Vivo

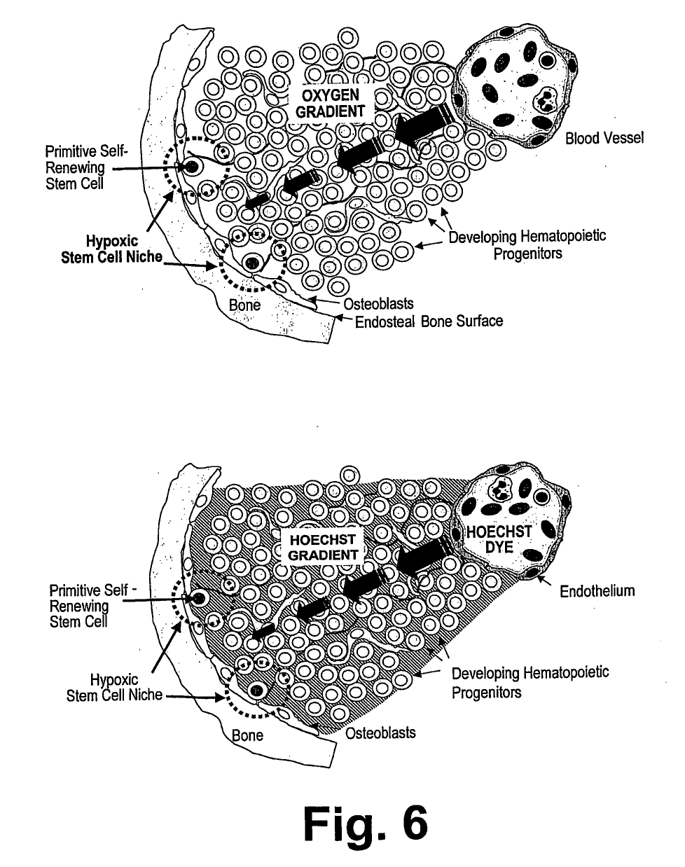

Several hypoxia-activated prodrugs have now been developed, among which the benzotriazine, Tirapazamine (TPZ), also known as SR4233, has been the most extensively studied and has therapeutic efficacy to the extent that it has now entered Phase II and III clinical trials in combination with radiotherapy and chemotherapy (Rischin et al., 2005; von Pawel et al., 2000). Under hypoxic conditions, TPZ is reduced to a benzotriazinyl radical and other reactive intermediates that ultimately leads to DNA double-strand breaks and cell death (FIG. 7). However, when oxygen is present, the TPZ radical is back-oxidized to the nontoxic parent compound (Brown and Wilson, 2004; Peters and Brown, 2002).

The present study therefore arose from the idea that HSCs might be rendered sensitive to TPZ treatments since many HSCs are hypoxic. Accordingly, age-matched (15-16 weeks old, for radiation/TPZ treatment and 9 weeks old for Busulfex treatment) groups of 3-4 male B6 recipient mice received either: 1) Saline alone, 2) TPZ (Sigma-Aldrich, St. Louis, Mo., Lot # 082H4029) dissolved in saline at 1.5 mg/ml and injected i.p. in 200 μL/10 g body weight at doses of 30 mg/kg daily over 4 days (total dose=120 mg/kg), or 3) Busulfex consisting of 6 mg/ml busulfan dissolved in N,N-dimethylacetamide 33% wt/wt and polyethylene glycol 400, 67% wt/wt (Orphan Medical, Inc., Minnetonka, Minn., Lot # 62161-005-31) diluted with sterile saline and injected i.p. in 100 μL/10 g body weight volumes at doses or 10 mg/kg daily over 2 days (total dose=20 mg/kg). Mice were given total body irradiation (TBI) at a dose rate of 94 cGy/min and total does of 400 cGy using ¹³⁷CS source (Gamma Cell 40, Atomic Energy of Canada, Ottawa, Canada).

At 24 hours after the last treatment, mice were sacrificed, the femori and tibias were removed, crushed in a morter and pestle, filtered and single cell suspensions of bone marrow cells in HBSS+ (Hank's balanced solution containing 2% FBS and 10 mM Hepes buffer, Gibco). The nucleated cell yield per femur was determined.

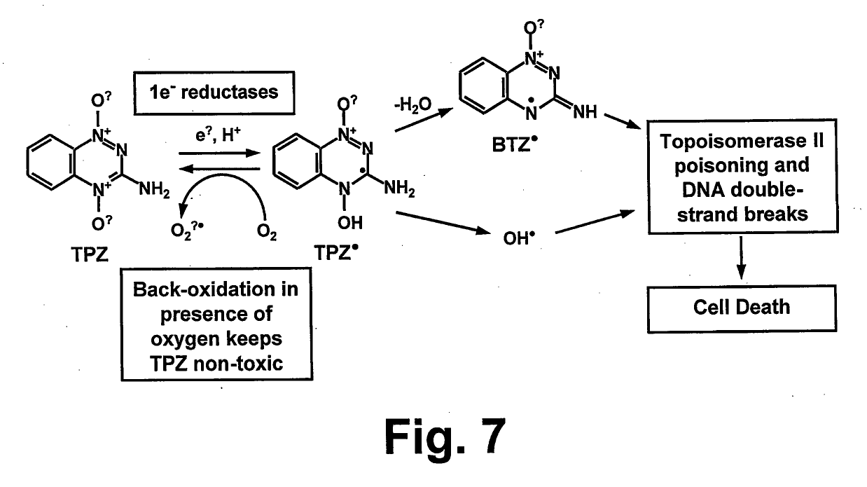

As shown in FIG. 8, evaluation of CAFC content in pooled bone marrow from mice treated in vivo with Tirapazamine (TPZ) showed higher depletion of late-versus early-forming CAFC subsets by this drug. This experiment shows how TPZ selectively depletes primitive CAFCS that correspond to the HSC subset. As the degree of depletion of the late-forming CAFC subsets (developing at 28 to 35 days in culture) in the recipient, following treatment with other types of agents, shows a strong correlation with the extent of long-term donor-type hematopoietic chimerism after bone marrow transplantation, (Down and Ploemacher, 1993, Exp. Hematol. 21:913-921; Down et al, 1994, Br. J. Cancer 1994, 70:611-616; Down et al., 1995 Blood 86:122-127; Westerhof G R et al, 2000, Cancer Res. 60:5470-5478; Ploemacher R E et al, 2004, Biology of Blood and Bone Marrow Transplantation 10: 236-245) then it is to be expected that TPZ treatment will facilitate engraftment of transplanted HSCs.

### Example 4

### Illustrations of Depleting Hypoxic Hematopoietic and Leukemic Stem Cells by Tirapazamine Treatment and Consequences for Donor Hematopoietic Stein Cell Engraftment and Eradication of Malignant Disease

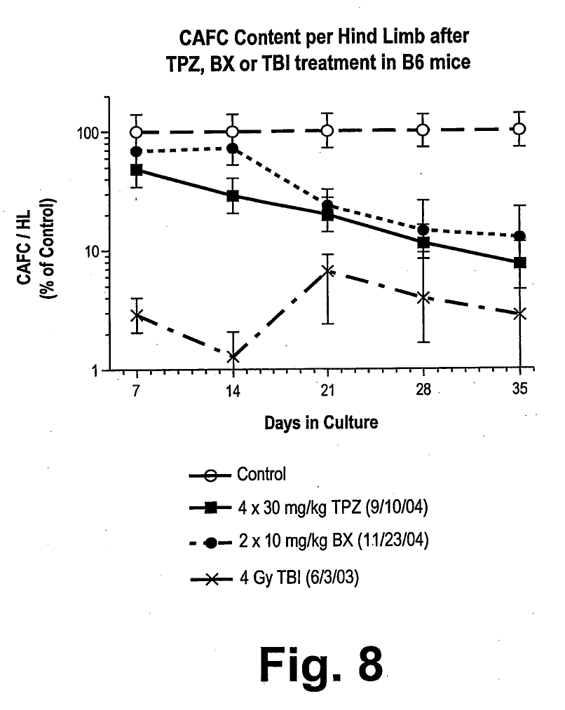

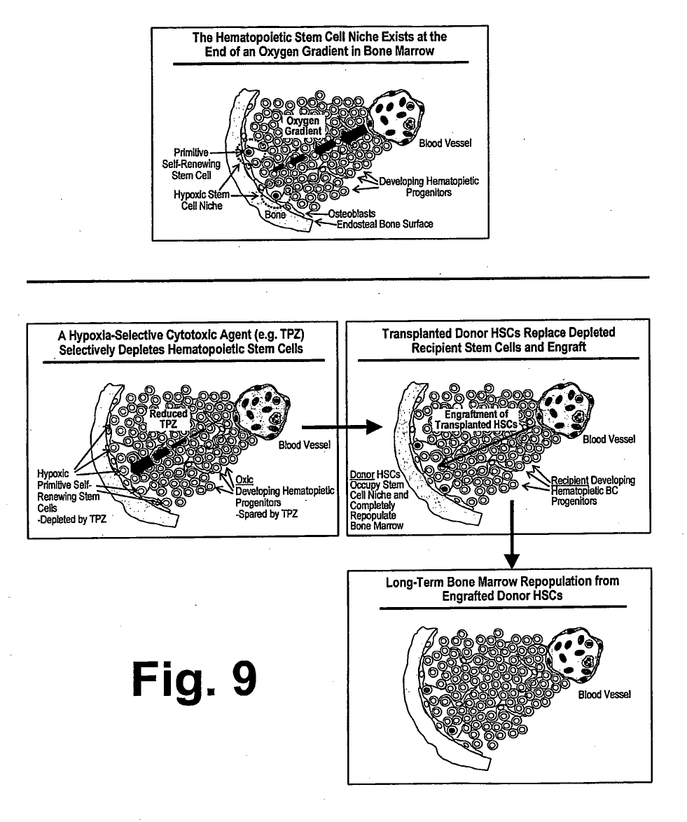

FIG. 9 gives a diagrammatic representation as to how tirapazime (TPZ) depletes hematopoietic cells within the bone marrow as an inverse relationship with the oxygen gradient whereby, HSCs are rendered more sensitive by virtue of their residence in a hypoxic microenvironmental niche. This scheme illustrates how TPZ facilitates engraftment of and repopulation by donor HSCs following transplantation. Since certain malignant stem cells (e.g. of leukemia) may reside in the same hypoxic niche, these stem cells will similarly be depleted after TPZ treatment and allow for eradication of the disease (FIG. 10).

### Example 5

### 5-Fluoronracil Treatment Perturbs the Hoechst Diffusion Gradient in the Bone Marrow and Leads to a Loss in the SP Phenotype

The anti-metabolite 5-fluorouracil (5-FU) is an established chemotherapeutic drug that has been one of the most extensively investigated agents with respect to its effect on the so hematopoietic system. The interest lies in the discriminatory effects of both in vivo and in vitro treatments in depleting cycling progenitor populations. David Harrison and colleagues (Harrison and Lerner, 1991) were among the first to pioneer the use of this drug to establish that bone marrow HSCs capable of long-term engraftment in irradiated recipients were resistant to 5-FU and therefore probably in a slow or non-cycling state under normal steady state conditions. This resistance, however, is dramatically lost when a second dose of 5-FU is delivered at 2 to 4 days after the first, leading to the conclusion that the ensuing period encompasses a homeostatic process whereby resting HSCs are prompted to enter active proliferation in response to loss of their ascendant progeny.

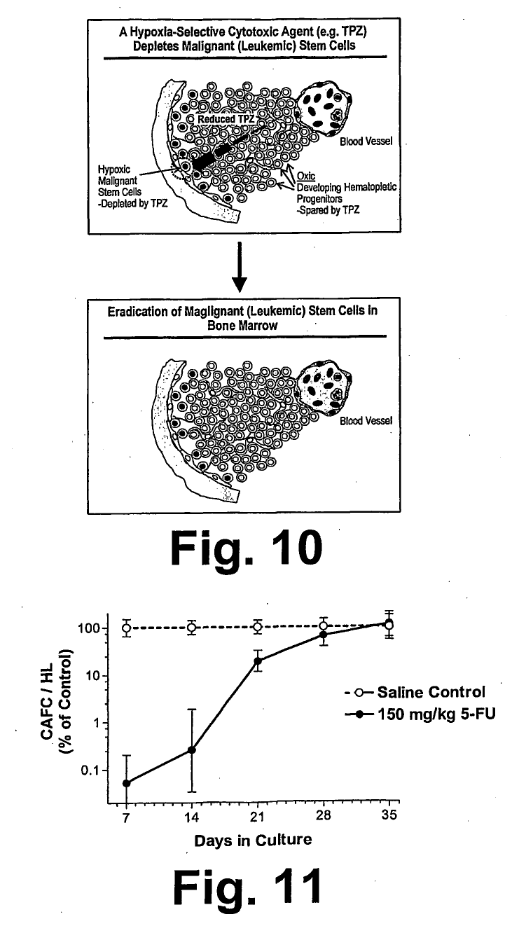

It has previously been shown that 5-FU can similarly deplete HSC following their stimulation with c-kit ligand (van Os et al., 1997). The selective toxicity of single dose 5-FU towards committed progenitors is also clearly shown by the marked depletion of early-forming CAFC subsets while the bone marrow content of the primitive late-forming CAFCs remain normal as exemplified in FIG. 11.

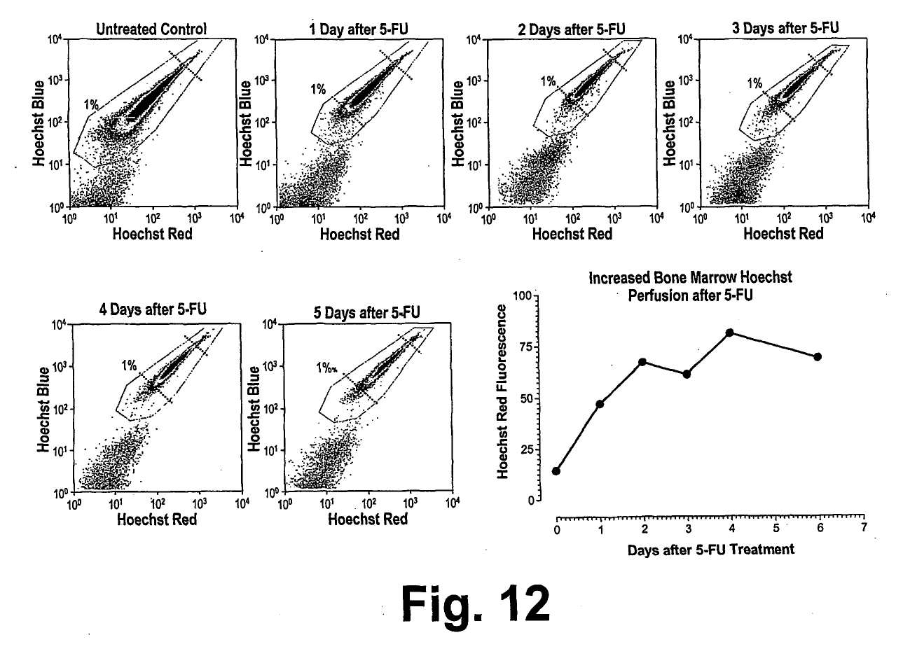

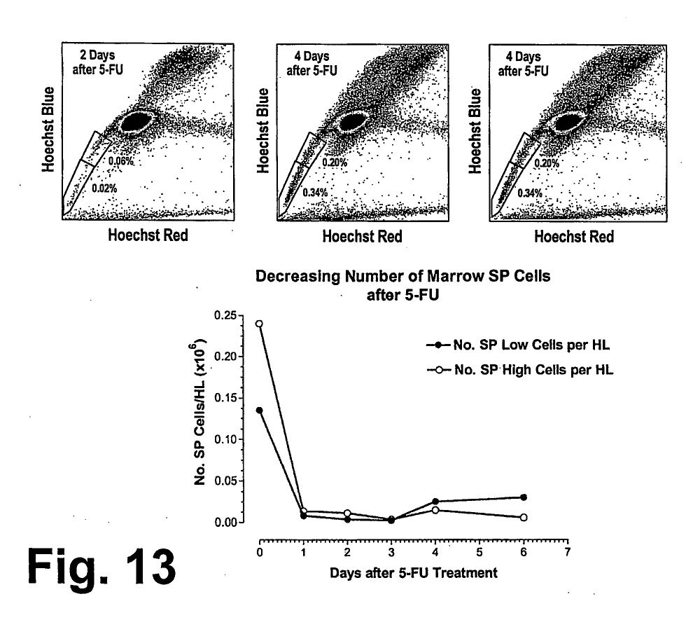

The well-described effect of 5-FU on stimulation of murine HSCs prompted the present study to determine how this relates to bone marrow perfusion of the Hoechst dye, as well as the number of cells exhibiting the SP phenotype. FIG. 12 shows how the Hoechst gradient is considerably shortened over a 6-day period after administering this drug. This feature appears to be co-incident with the dramatic decrease in the percent and overall marrow content of SP cells as shown in FIG. 13.

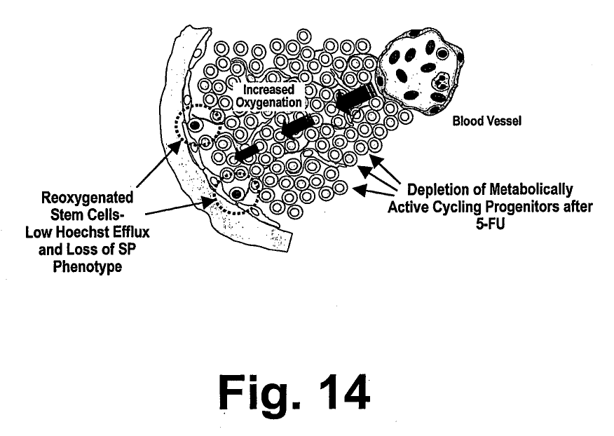

As the number of functional HSCs remains unaffected by 5-FU, it can be concluded that these cells no longer have the ability to efflux Hoechst and therefore lose their typical SP characteristic. The increased fluorescence and shortening of the Hoechst gradient on i.v. infusion of this dye is suggestive of improved oxygenation of HSCs as diagrammatically illustrated in FIG. 14. This shows that the prior 5-FU treatment has the effect of destroying oxygen-consuming and metabolically active progenitors allowing reoxygenation of the HSC niche, a phenomenon that is not too dissimilar from the situation that occurs during radiation treatment of solid tumors (Begg et al., 1969; Field et al., 1968; Howes, 1969; Van Putten and Kallman, 1968).

### REFERENCES

1. Abkowitz, J. L., Robinson, A. E., Kale, S., Long, M. W., and Chen, J. (2003). Mobilization of hematopoietic stem cells during homeostasis and after cytokine exposure. Blood 102, 1249-1253.
2. Airley, R., Loncaster, J., Davidson, S., Bromley, M., Roberts, S., Patterson, A., Hunter, R., Stratford, I., and West, C. (2001).Glucose transporter glut-1 expression correlates with tumor hypoxia and predicts metastasis-free survival in advanced carcinoma of the cervix. Clin Cancer Res 7, 928-934.
3. Airley, R. E., Loncaster, J., Raleigh, J. A., Harris, A. L., Davidson, S. E., Hunter, R. D., West, C. M., and Stratford, I. J. (2003). GLUT-1 and CAIX as intrinsic markers of hypoxia in carcinoma of the cervix: relationship to pimonidazole binding. Int J Cancer 104, 85-91.
4. Albini, A., Morini, M., D'Agostini, F., Ferrari, N., Campelli, F., Arena, G., Noonan, D. M., Pesce, C., and De Flora, S. (2001). Inhibition of angiogenesis-driven Kaposi's sarcoma tumor growth in nude mice by oral N-acetylcysteine. Cancer Res 61, 8171-8178.
5. Ames, B. N., Shigenaga, M. K., and Gold, L. S. (1993). DNA lesions, inducible DNA repair, and cell division: three key factors in mutagenesis and carcinogenesis. Environ Health Perspect 101 Suppl 5, 35-44.
6. Amai, F., Hirao, A., Ohmura, M., Sato, H., Matsuoka, S., Takubo, K., Ito, K., Koh, G. Y., and Suda, T. (2004). Tie2/angiopoietin-1 signaling regulates hematopoietic stem cell quiescence in the bone marrow niche. Cell 118, 149-161.
7. Begg, A. C., Denekamp, J., and Howes, A. E. (1969). Re-oxygenation of tumours after irradiation and cell population kinetics. J Physiol 202, 16P-17P.
8. Bernsen, H. J., Rijken, P. F., Peters, H., Raleigh, J. A., Jeuken, J. W., Wesseling, P., and van der Kogel, A. J. (2000). Hypoxia in a human intracerebral glioma model. J Neurosurg 93, 449-454.
9. Bertoncello, I., Bradley, T. R., Hodgson, G. S., and Dunlop, J. M. (1991). The resolution, enrichment, and organization of normal bone marrow high proliferative potential colony-forming cell subsets on the basis of rhodamine-123 fluorescence. Exp Hematol 19, 174-178.
10. Bodine, D. M., Seidel, N. E., and Orlic, D. (1996). Bone marrow collected 14 days after in vivo administration of granulocyte colony-stimulating factor and stem cell factor to mice has 10-fold more repopulating ability than untreated bone marrow. Blood 88, 89-97.
11. Bodine, D. M., Seidel, N. E., Zsebo, K. M., and Orlic, D. (1993). In vivo administration of stem cell factor to mice increases the absolute number of pluripotent hematopoietic stem cells. Blood 82, 445-455.
12. Briddell, R. A., Hartley, C. A., Smith, K. A., and McNiece, I. K. (1993). Recombinant rat stem cell factor synergizes with recombinant human granulocyte colony-stimulating factor in vivo in mice to mobilize peripheral blood progenitor cells that have enhanced repopulating potential. Blood 82, 1720-1723.
13. Brown, J. M. (1979). Evidence for acutely hypoxic cells in mouse tumours, and a possible mechanism of reoxygenation. Br J Radiol 52, 650-656.
14. Brown, J. M., and Wilson, W. R. (2004). Exploiting tumour hypoxia in cancer treatment. Nat Rev Cancer 4, 437-447.
15. Broxmeyer, H. E., Cooper, S., Hangoc, G., Gao, J. L., and Murphy, P. M. (1999). Dominant myelopoietic effector functions mediated by chemokine receptor CCR1. J Exp Med 189, 1987-1992.
16. Bunting, K. D. (2002). ABC transporters as phenotypic markers and functional regulators of stem cells. Stem Cells 20, 11-20.
17. Calvi, L. M., Adams, G. B., Weibrecht, K. W., Weber, J. M., Olson, D. P., Knight, M. C., Martin, R. P., Schipani, E., Divieti, P., Bringhurst, F. R., et al. (2003). Osteoblastic cells regulate the haematopoietic stem cell niche. Nature 425, 841-846.
18. Ceradimi, D. J., Kulkarni, A. R., Callaghan, M. J., Tepper, O. M., Bastidas, N., Kleinman, M. E., Capla, J. M., Galiano, R. D., Levine, J. P., and Gurtner, G. C. (2004). Progenitor cell trafficking is regulated by hypoxic gradients through HIF-1 induction of SDF-1. Nat Med 10, 858-864.
19. Chaplin, D. J., Olive, P. L., and Durand, R. E. (1987). Intermittent blood flow in a murine tumor: radiobiological effects. Cancer Res 47, 597-601.
20. Cheshier, S. H., Morrison, S. J., Liao, X., and Weissman, I. L. (1999). In vivo proliferation and cell cycle kinetics of long-term self-renewing hematopoietic stem cells. Proc Natl Acad Sci USA 96, 3120-3125.
21. Chow, D. C., Wenning, L. A., Miller, W. M., and Papoutsakis, E. T. (2001). Modeling pO(2) distributions in the bone marrow hematopoietic compartment. II. Modified Kroghian models. Biophys J 81, 685-696.
22. Cipolleschi, M. G., Dello Sbarba, P., and Olivotto, M. (1993). The role of hypoxia in the maintenance of hematopoietic stem cells. Blood 82, 2031-2037.
23. Colvin, G. A., Lambert, J. F., Abedi, M., Dooner, M. S., Demers, D., Moore, B. E., so Greer, P., Aliotta, J. M., Pimentel, J., Cerny, J., et al. (2004). Differentiation hotspots: the deterioration of hierarchy and stochasm. Blood Cells Mol Dis 32, 34-41.
24. D'Andrea, A. D., and Grompe, M. (2003). The Fanconi anaemia/BRCA pathway. Nat Rev Cancer 3, 23-34.
25. Danet, G. H., Pan, Y., Luongo, J. L., Bonnet, D. A., and Simon, M. C. (2003). Expansion of human SCID-repopulating cells under hypoxic conditions. J Clin Invest 112, 126-135.
26. D'Ippolito, G., Diabira, S., Howard, G. A., Menei, P., Roos, B. A., and Schiller, P. C. (2004). Marrow-isolated adult multilineage inducible (MIAMI) cells, a unique population of postnatal young and old human cells with extensive expansion and differentiation potential. J Cell Sci 117, 2971-2981.
27. Down, J. D., Boudewijn, A., Dillingh, J. H., Fox, B. W., and Ploemacher, R. E. (1994). Relationships between ablation of distinct haematopoietic cell subsets and the development of donor bone marrow engraftment following recipient pretreatment with different alkylating drugs. Br J Cancer 70, 611-616.
28. Down, J. D., Boudewijn, A., van Os, R., Thames, H. D., and Ploemacher, R. E. (1995). Variations in radiation sensitivity and repair among different hematopoietic stem cell subsets following fractionated irradiation. Blood 86, 122-127.
29. Down, J. D., de Haan, G., Dillingh, J. H., Dontje, B., and Nijhof, W. (1997). Stem cell factor has contrasting effects in combination with 5-fluorouracil or total-body irradiation on frequencies of different hemopoietic cell subsets and engraftment of transplanted bone marrow. Radiat Res 147, 680-685.
30. Down, J. D., and Ploemacher, R. E. (1993). Transient and permanent engraftment potential of murine hematopoietic stem cell subsets: differential effects of host conditioning with gamma radiation and cytotoxic drugs. Exp Hematol 21, 913-921.
31. Duncan, A. W., Rattis, F. M., DiMascio, L. N., Congdon, K. L., Pazianos, G., Zhao, C., Yoon, K., Cook, J. M., Willert, K., Gaiano, N., and Reya, T. (2005). Integration of Notch and Wnt signaling in hematopoietic stem cell maintenance. Nat Immunol 6, 314-322.
32. Durand, R. E., Chaplin, D. J., and Olive, P. L. (1990). Cell sorting with Hoechst or carbocyanine dyes as perfusion probes in spheroids and tumors. Methods Cell Biol 33, 509-518.
33. Durand, R. E., and Raleigh, J. A. (1998). Identification of nonproliferating but viable hypoxic tumor cells in vivo. Cancer Res 58, 3547-3550.
34. Field, S. B., Jones, T., and Thomlinson, R. H. (1968). The relative effects of fast neutrons and x rays on tumour and normal tissue in the rat. II. Fractionation: recovery and reoxygenation. Br J Radiol 41, 597-607.
35. Frassoni, F., Testa, N. G., and Lord, B. I. (1982). The relative spatial distribution of erythroid progenitor cells (BFUe and CFUe) in the normal mouse femur. Cell Tissue Kinet 15, 447-455.
36. Giaccia, A. J., Auger, B. A., Koong, A., Terris, D. J., Minchinton, A. I., Hahn, G. M., and Brown, J. M. (1992). Activation of the heat shock transcription factor by hypoxia in normal and tumor cell lines in vivo and in vitro. Int J Radiat Oncol Biol Phys 23, 891-897.
37. Goodell, M. A., Brose, K., Paradis, G., Conner, A. S., and Mulligan, R. C. (1996). Isolation and functional properties of murine hematopoietic stem cells that are replicating in vivo. J Exp Med 183, 1797-1806.
38. Hale, L. P., Braun, R. D., Gwinn, W. M., Greer, P. K., and Dewhirst, M. W. (2002). Hypoxia in the thymus: role of oxygen tension in thymocyte survival. Am J Physiol Heart Circ Physiol 282, H1467-1477.
39. Hammond, E. M., Dorie, M. J., and Giaccia, A. J. (2004). Inhibition of ATR leads to increased sensitivity to hypoxia/reoxygenation. Cancer Res 64, 6556-6562.
40. Hammond, E. M., and Giaccia, A. J. (2004). The role of ATM and ATR in the cellular response to hypoxia and re-oxygenation. DNA Repair (Amst) 3, 1117-1122.
41. Harrison, D. E. (1980). Competitive repopulation: a new assay for long-term stem cell functional capacity. Blood 55, 77-81.
42. Harrison, D. E., and Lerner, C. P. (1991). Most primitive hematopoietic stem cells are stimulated to cycle rapidly after treatment with 5-fluorouracil. Blood 78, 1237-1240.
43. Harrison, D. E., Zsebo, K. M., and Astle, C. M. (1994). Splenic primitive hematopoietic stem cell (PHSC) activity is enhanced by steel factor because of PHSC proliferation. Blood 83, 3146-3151.
44. Hierck, B. P., Iperen, L. V., Gittenberger-De Groot, A. C., and Poelmann, R. E. (1994). Modified indirect immunodetection allows study of murine tissue with mouse monoclonal antibodies. J Histochem Cytochem 42, 1499-1502.
45. Hoskin, P. J., Sibtain, A., Daley, F. M., and Wilson, G. D. (2003). GLUT1 and CAIX as intrinsic markers of hypoxia in bladder cancer: relationship with vascularity and proliferation as predictors of outcome of ARCON. Br J Cancer 89, 1290-1297.
46. Howes, A. E. (1969). An estimation of changes in the proportions and absolute numbers of hypoxic cells after irradiation of transplanted C3H mousemammary tumours. Br J Radiol 42, 441-447.
47. Ito, H., Takeuchi, Y., Shaffer, J., and Sykes, M. (2004a). Local irradiation enhances congenic donor pluripotent hematopoietic stem cell engraftment similarly in irradiated and nonirradiated sites. Blood 103, 1949-1954.
48. Ito, K., Hirao, A., Arai, F., Matsuoka, S., Takubo, K., Hamaguchi, I., Nomiyama, K., Hosokawa, K., Sakurada, K., Nakagata, N., et al. (2004b). Regulation of oxidative stress by ATM is required for self-renewal of haematopoietic stem cells. Nature 431, 997-1002.
49. Ivan, M., Kondo, K., Yang, H., Kim, W., Valiando, J., Olli, M., Salic, A., Asara, J. M., Lane, W. S., and Kaelin, W. G., Jr. (2001). HIFalpha targeted for VHL-mediated destruction by proline hydroxylation: implications for O2 sensing. Science 292, 464-468.
50. Ivanovic, Z., Dello Sbarba, P., Trimoreau, F., Faucher, J. L., and Praloran, V. (2000). Primitive human HPCs are better maintained and expanded in vitro at 1 percent oxygen than at 20 percent. Transfusion 40, 1482-1488.
51. Jaakkola, P., Mole, D. R., Tian, Y. M., Wilson, M. I., Gielbert, J., Gaskell, S. J., Kriegsheim, A., Hebestreit, H. F., Mukheiji, M., Schofield, C. J., et al. (2001). Targeting of HIF-alpha to the von Hippel-Lindau ubiquitylation complex by O2-regulated prolyl hydroxylation. Science 292, 468-472.
52. Jiang, Y., Jahagirdar, B. N., Reinhardt, R. L., Schwartz, R. E., Keene, C. D., Ortiz-Gonzalez, X. R., Reyes, M., Lenvik, T., Lund, T., Blackstad, M., et al. (2002). Pluripotency of mesenchymal stem cells derived from adult marrow. Nature 418, 41-49.
53. Jones, R. J., Wagner, J. E., Celano, P., Zicha, M. S., and Sharkis, S. J. (1990). Separation of pluripotent haematopoietic stem cells from spleen colony-forming cells. Nature 347, 188-189.
54. Kim, M., Turnquist, H., Jackson, J., Sgagias, M., Yan, Y., Gong, M., Dean, M., Sharp, J. G., and Cowan, K. (2002). The multidrug resistance transporter ABCG2 (breast cancer resistance protein 1) effluxes Hoechst 33342 and is overexpressed in hematopoietic stem cells. Clin Cancer Res 8, 22-28.
55. Kogure, K., Watson, B. D., Busto, R., and Abe, K. (1982). Potentiation of lipid peroxides by ischemia in rat brain. Neurochem Res 7, 437-454.
56. Krishnamurthy, P., Ross, D. D., Nakanishi, T., Bailey-Dell, K., Zhou, S., Mercer, K. E., Sarkadi, B., Sorrentino, B. P., and Schuetz, J. D. (2004). The stem cell marker Bcrp/ABCG2 enhances hypoxic cell survival through interactions with heme. J Biol Chem 279, 24218-24225.
57. Li, C., and Jackson, R. M. (2002). Reactive species mechanisms of cellular hypoxia-reoxygenation injury. Am J Physiol Cell Physiol 282, C227-241.
58. Liu, F., Poursine-Laurent, J., and Link, D. C. (2000). Expression of the G-CSF receptor on hematopoietic progenitor cells is not required for their mobilization by G-CSF. Blood 95, 3025-3031.
59. Loncaster, J. A., Harris, A. L., Davidson, S. E., Logue, J. P., Hunter, R. D., Wycoff, C. C., Pastorek, J., Ratcliffe, P. J., Stratford, I. J., and West, C. M. (2001). Carbonic anhydrase (CA IX) expression, a potential new intrinsic marker of hypoxia: correlations with tumor oxygen measurements and prognosis in locally advanced carcinoma of the cervix. Cancer Res 61, 6394-6399.
60. Lord, B. I. (1990). The architecture of bone marrow cell populations. Int J Cell Cloning 8, 317-331.
61. Lord, B. I., Testa, N. G., and Hendry, J. H. (1975). The relative spatial distributions of CFUs and CFUc in the normal mouse femur. Blood 46, 65-72.
62. Maloney, M. A., Lamela, R. A., Dorie, M. J., and Patt, H. M. (1978). Concentration gradient of blood stem cells in mouse bone marrow—an open question. Blood 51, 521-525.
63. Mason, T. M., Lord, B. I., and Hendry, I. H. (1989). The development of spatial distributions of CFU-S and in-vitro CFC in femora of mice of different ages. Br J Haematol 73, 455-461.
64. Matsuzaki, Y., Kinjo, K., Mulligan, R. C., and Okano, H. (2004). Unexpectedly efficient homing capacity of purified murine hematopoietic stem cells. Immunity 20, 87-93.
65. McNiece, I. K., Briddell, R. A., Hartley, C. A., Smith, K. A., and Andrews, R. G. (1993). Stein cell factor enhances in vivo effects of granulocyte colony stimulating factor for stimulating mobilization of peripheral blood progenitor cells. Stem Cells 11 Suppl 2, 36-41.
66. Migliaccio, G., Migliaccio, A. R., Valinsky, J., Langley, K., Zsebo, K., Visser, J. W., and Adamson, J. W. (1991). Stem cell factor induces proliferation and differentiation of highly enriched murine hematopoietic cells. Proc Natl Acad Sci USA 88, 7420-7424.
67. Minchinton, A. I., and Brown, J. M. (1992). Enhancement of the cytotoxicity of SR 4233 to normal and malignant tissues by hypoxic breathing. Br J Cancer 66, 1053-1058.
68. Minchinton, A. I., Lemmon, M. J., Tracy, M., Pollart, D. J., Martinez, A. P., Tosto, L. M., and Brown, J. M. (1992). Second-generation 1,2,4-benzotriazine 1,4-di-N-oxide bioreductive anti-tumor agents: pharmacology and activity in vitro and in vivo. Int J Radiat Oncol Biol Phys 22, 701-705.
69. Molineux, G., Migdalska, A., Haley, J., Evans, G. S., and Dexter, T. M. (1994). Total marrow failure induced by pegylated stem-cell factor administered before 5-fluorouracil. Blood 83, 3491-3499.
70. Molineux, G., Pojda, Z., Hampson, I. N., Lord, B. I., and Dexter, T. M. (1990). Transplantation potential of peripheral blood stem cells induced by granulocyte colony-stimulating factor. Blood 76, 2153-2158.
71. Morrison, S. J., Wright, D. E., and Weissman, I. L. (1997). Cyclophosphamide/granulocyte colony-stimulating factor induces hematopoietic stem cells to proliferate prior to mobilization. Proc Natl Acad Sci USA 94, 1908-1913.
72. Neben, S., Marcus, K., and Mauch, P. (1993). Mobilization of hematopoietic stem and progenitor cell subpopulations from the marrow to the blood of mice following cyclophosphamide and/or granulocyte colony-stimulating factor. Blood 81, 1960-1967.
73. Neben, S., Redfearn, W. J., Parra, M., Brecher, G., and Pallavicini, M. G. (1991). Short- and long-term repopulation of lethally irradiated mice by bone marrow stem cells enriched on the basis of light scatter and Hoechst 33342 fluorescence. Exp Hematol 19, 958-967.
74. Nilsson, S. K., Johnston, H. M., and Coverdale, J. A. (2001). Spatial localization of transplanted hemopoietic stem cells: inferences for the localization of stem cell niches. Blood 97, 2293-2299.
75. Olive, P. L., Aquino-Parsons, C., MacPhail, S. H., Liao, S. Y., Raleigh, J. A., Lerman, M. I., and Stanbridge, E. J. (2001). Carbonic anhydrase 9 as an endogenous marker for hypoxic cells in cervical cancer. Cancer Res 61, 8924-8929.
76. Olive, P. L., Durand, R. E., Raleigh, J. A., Luo, C., and Aquino-Parsons, C. (2000). Comparison between the comet assay and pimonidazole binding for measuring tumour hypoxia. Br J Cancer 83, 1525-1531.
77. Olive, P. L., Luo, C. M., and Banath, J. P. (2002). Local hypoxia is produced at sites of intratumour injection. Br J Cancer 86, 429-435.
78. Olmsted-Davis, E. A., Gugala, Z., Camargo, F., Gannon, F. H., Jackson, K., Kienstra, K. A., Shine, H. D., Lindsey, R. W., Hirschi, K. K., Goodell, M. A., et al. (2003). Primitive adult hematopoietic stem cells can function as osteoblast precursors. Proc Natl Acad Sci USA 100, 15877-15882.
79. Papadopoulotu, M. V., Ji, M., Ji, X., and Bloomer, W. D. (2002). Therapeutic advantage from combining 5-fluorouracil with the hypoxia-selective cytotoxin NLCQ-1 in vivo; comparison with tirapazamine. Cancer Chemother Pharmacol 50, 291-298.
80. Parmar, K., Sauk-Schubert, C., Burdick, D., Handley, M., and Mauch, P. (2003). Sca+CD34-murine side population cells are highly enriched for primitive stem cells. Exp Hematol 31, 244-250.
81. Parmar, K., Burdick, D., Ehtier, M., Clyne, J., and Mauch, P. (2005). Murine side population cells contain hematopoietic stem cell activity in mobilized blood. Stem Cells and Development (in press).
82. Peters, K. B., and Brown, J. M. (2002). Tirapazamine: a hypoxia-activated topoisomerase II poison. Cancer Res 62, 5248-5253.
83. Pittenger, M. F., Mackay, A. M., Beck, S. C., Jaiswal, R. K., Douglas, R., Mosca, J. D., Moorman, M. A., Simonetti, D. W., Craig, S., and Marshak, D. R. (1999). Multilineage potential of adult human mesenchymal stem cells. Science 284, 143-147.
84. Ploemacher, R. E., and Brons, N. H. (1988). In vivo proliferative and differential properties of murine bone marrow cells separated on the basis of rhodamine-123 retention. Exp Hematol 16, 903-907.
85. Ploemacher, R. E., and Brons, R. H. (1989). Separation of CFU-S from primitive cells responsible for reconstitution of the bone marrow hemopoietic stem cell compartment following irradiation: evidence for a pre-CFU-S cell. Exp Hematol 17, 263-266.
86. Ploemacher, R. E., Johnson, K. W., Rombouts, E. J., Etienne, K., Westerhof, G. R., Baumgart, J., white-Scharf, M. E., and Down, J. D. (2004). Addition of treosulfan to a nomnyeloablative conditioning regimen results in enhanced chimerism and immunologic tolerance in an experimental allogeneic bone marrow transplant model. Biol Blood Marrow Transplant 10, 236-245.
87. Ploemacher, R. E., van der Sluijs, J. P., van Beurden, C. A., Baert, M. R., and Chan, P. L. (1991). Use of limiting-dilution type long-term marrow cultures in frequency analysis of marrow-repopulating and spleen colony-forming hematopoietic stem cells in the mouse. Blood 78, 2527-2533.
88. Ploemacher, R. E., van der Sluijs, J. P., Voennan, J. S., and Brons, N. H. (1989). An in vitro limiting-dilution assay of long term repopulating hematopoietic stem cells in the mouse. Blood 74, 2755-2763.
89. Reyes, M., Dudek, A., Jahagirdar, B., Koodie, L., Marker, P. H., and Verfaillie, C. M. (2002). Origin of endothelial progenitors in human postnatal bone marrow. J Clin Invest 109, 337-346.
90. Rischin, D., Peters, L., Fisher, R., Macann, A., Denham, J., Poulsen, M., Jackson, M., Kenny, L., Penniment, M., Corry, J., et al. (2005). Tirapazamine, Cisplatin, and Radiation versus Fluorouracil, Cisplatin, and Radiation in patients with locally advanced head and neck cancer: a randomized phase II trial of the Trans-Tasman Radiation Oncology Group (TROG 98.02). J Clin Oncol 23, 79-87.
91. Scharenberg, C. W., Harkey, M. A., and Torok-Storb, B. (2002). The ABCG2 transporter is an efficient Hoechst 33342 efflux pump and is preferentially expressed by immature human hematopoietic progenitors. Blood 99, 507-512.
92. Schofield, R. (1978). The relationship between the spleen colony-forming cell and the hemopoietic stem cell. Blood Cells 4, 7-25.
93. Semenza, G. L. (1998). Hypoxia-inducible factor 1: master regulator of O2 homeostasis. Curr Opin Genet Dev 8, 588-594.
94. Semenza, G. L. (2000). Expression of hypoxia-inducible factor 1: mechanisms and consequences. Biochem Pharmacol 59, 47-53.
95. Semenza, G. L. (2003). Targeting HIF-1 for cancer therapy. Nat Rev Cancer 3, 721-732.
96. Sharabi, Y., and Sachs, D. H. (1989). Engraftment of allogeneic bone marrow following administration of anti-T cell monoclonal antibodies and low-dose irradiation. Transplant Proc 21, 233-235.
97. Siim, B. G., Pruijn, F. B., Sturman, J. R., Hogg, A., Hay, M. P., Brown, J. M., and Wilson, W. R. (2004). Selective potentiation of the hypoxic cytotoxicity of tirapazamine by its 1-N-oxide metabolite SR 4317. Cancer Res 64, 736-742.
98. Spangrude, G. J., Heimfeld, S., and Weissman, I. L. (1988a). Purification and characterization of mouse hematopoietic stem cells. Science 241, 58-62.
99. Spangrude, G. J., Muller-Sieburg, C. E., Heimfeld, S., and Weissman, I. L. (1988b). Two rare populations of mouse Thy-1lo bone marrow cells repopulate the thymus. J Exp Med 167, 1671-1683.
100. Spangrude, G. J., and Scollay, R. (1990). Differentiation of hematopoietic stem cells in irradiated mouse thymic lobes. Kinetics and phenotype of progeny. J Immunol 145, 3661-3668.
101. Stadtman, E. R. (1992). Protein oxidation and aging. Science 257, 1220-1224.
102. Stier, S., Ko, Y., Forkert, R., Lutz, C., Neuhaus, T., Gruenewald, E., Dombkowski, D., Rittling, S R., Scadden, D T. (2004). Matrix glycoprotein osteopontin is a stem cell niche constituent that constrains the hematopoietic stem cell pool size. Blood 104(11):191A, abstract # 664, 46th ASH meeting held at San Diego, Calif.
103. Sun, S., Guo, Z., Xiao, X., Liu, B., Liu, X., Tang, P. H., and Mao, N. (2003). Isolation of mouse marrow mesenchymal progenitors by a novel and reliable method. Stem Cells 21, 527-535.
104. Taylor, G., Lehrer, M. S., Jensen, P. J., Sun, T. T., and Lavker, R. M. (2000). Involvement of follicular stem cells in forming not only the follicle but also the epidermis. Cell 102, 451-461.
105. Thomlinson, R. H., and Gray, L. H. (1955). The histological structure of some human lung cancers and the possible implications for radiotherapy. Br J Cancer 9, 539-549.
106. Tice, R. R., Agurell, E., Anderson, D., Burlinson, B., Hartmann, A., Kobayashi, H., Miyamae, Y., Rojas, B., Ryn, J. C., and Sasaki, Y. F. (2000). Single cell gel/comet assay: guidelines for in vitro and in vivo genetic toxicology testing. Environ Mol Mutagen 35, 206-221.
107. van Laarhoven, H. W., Bussink, J., Lok, J., Punt, C. J., Heerschap, A., and van Der Kogel, A. J. (2004). Effects of nicotinamide and carbogen in different murine colon carcinomas: immunohistochemical analysis of vascular architecture and microenvironmental parameters. Int J Radiat Oncol Biol Phys 60, 310-321.
108. van Os, R., Dawes, D., Mislow, J. M., Witsell, A., and Mauch, P. M. (1997). Host conditioning with 5-fluorouracil and kit-ligand to provide for long-term bone marrow engraftment. Blood 89, 2376-2383.
109. Van Putten, L. M., and Kallman, R. F. (1968). Oxygenation status of a transplantable tunor during fractionated radiation therapy. J Natl Cancer Inst 40, 441-451.
110. Visser, J. W., de Vries, P., Hogeweg-Platenburg, M. G., Bayer, J., Schoeters, G., van den Heuvel, R., and Mulder, D. H. (1991). Culture of hematopoietic stem cells purified from murine bone marrow. Semin Hematol 28, 117-125.
111. von Pawel, J., von Roemeling, R., Gatzeineier, U., Boyer, M., Elisson, L. O., Clark, P., Talbot, D., Rey, A., Butler, T. W., Hirsh, V., et al. (2000). Tirapazamine plus cisplatin versus cisplatin in advanced non-small-cell lung cancer: A report of the international CATAPULT I study group. Cisplatin and Tirapazamine in Subjects with Advanced Previously Untreated Non-Small-Cell Lung Tumors. J Clin Oncol 18, 1351-1359.
112. Wang, D., Christensen, K., Chawla, K., Xiao, G., Krebsbach, P. H., and Franceschi, R. T. (1999). Isolation and characterization of MC3T3-E1 preosteoblast subclones with distinct in vitro and in vivo differentiation/mineralization potential. J Bone Miner Res 14, 893-903.
113. Wang, G. L., Jiang, B. H., Rue, E. A., and Semenza, G. L. (1995). Hypoxia-inducible factor 1 is a basic-helix-loop-helix-PAS heterodimer regulated by cellular O2 tension. Proc Natl Acad Sci USA 92, 5510-5514.
114. Weissman, I. L., Anderson, D. J., and Gage, F. (2001). Stem and progenitor cells: origins, phenotypes, lineage commitments, and transdifferentiations. Annu Rev Cell Dev Biol 17, 387-403.
115. Westerhof, G. R., Ploemacher, R. E., Boudewijn, A., Blokland, I., Dillingh, J. H., McGown, A. T., Hadfield, J. A., Dawson, M. J., and Down, J. D. (2000). Comparison of different busulfan analogues for depletion of hematopoietic stem cells and promotion of donor-type chimerism in murine bone marrow transplant recipients. Cancer Res 60, 5470-5478.
116. Wright, D. E., Cheshier, S. H., Wagers, A. J., Randall, T. D., Christensen, J. L., and Weissman, I. L. (2001a). Cyclophosphamide/granulocyte colony-stimulating factor causes selective mobilization of bone marrow hematopoietic stem cells into the blood alter M phase of the cell cycle. Blood 97, 2278-2285.
117. Wright, D. E., Wagers, A. J., Gulati, A. P., Johnson, F. L., and Weissman, I. L. (2001b). Physiological migration of hematopoietic stem and progenitor cells. Science 294, 1933-1936.
118. Zeman, E. M., Brown, J. M., Lemmon, M. J., Hirst, V. K., and Lee, W. W. (1986). SR-4233: a new bioreductive agent with high selective toxicity for hypoxic mammalian cells. Int J Radiat Oncol Biol Phys 12, 1239-1242.
119. Zhang, J., Niu, C., Ye, L., Huang, H., He, X., Tong, W. G., Ross, J., Haug, J., Johnson, T., Feng, J. Q., et al. (2003). Identification of the haematopoietic stem cell niche and control of the niche size. Nature 425, 836-841.
120. Zhang, X., Li, J., Sejas, D. P., and Pang, Q. (2005a). The ATM/p53/p21 pathway influences cell fate decision between apoptosis and senescence in reoxygenated hematopoietic progenitor cells. J Biol Chem 280, 19635-19640.
121. Zhang, X., Li, J., Sejas, D. P., and Pang, Q. (2005b). Hypoxia-reoxygenation induces premature senescence in FA bone marrow hematopoietic cells. Blood.
122. Zhou, S., Schuetz, J. D., Bunting, K. D., Colapietro, A. M., Sampath, J., Morris, J. J., Lagutina, I., Grosveld, G. C., Osawa, M., Nakauchi, H., and Sorrentino, B. P. (2001). The ABC transporter Bcrp1/ABCG2 is expressed in a wide variety of stem cells and is a molecular determinant of the side-population phenotype. Nat Med 7, 1028-1034.

1. A method of selectively depleting hematopoietic stem cells within the bone marrow comprising contacting the cells in vivo with an agent that selectively kills hypoxic cells, such that the cells are depleted.

2. A method of engrafting donor hematopoietic stem cells in the bone marrow of a host subject comprising:
administering to the subject an agent that selectively kills hypoxic cells, such that hematopoietic stem cells in the subject are depleted; and
administering hematopoietic stem cells from a donor subject.

3. A method of treating a cancer within the bone marrow of a host subject comprising:
administering to the subject an agent that selectively kills hypoxic cells, such that the cells in the subject are depleted; and
administering hematopoietic stem cells from a donor subject, such that the cancer is treated.

4. The method of claim 3 wherein the hypoxic cells comprise cancer cells that have metastasized to the bone marrow.

5. The method of claim 4 wherein the hypoxic cells comprise neuroblastoma cells or breast carcinoma cells.

6. (canceled)

7. The method of claim 3 wherein the cancer is a hematological cancer selected from the group consisting of leukemia and lymphoma.

8. (canceled)

9. The method of any one of claims 1-3, wherein the agent is administered to a subject prior to administration of donor cells selected from the group consisting of donor bone marrow, donor cytokine mobilized peripheral blood, and donor umbilical cord blood.

10-11. (canceled)

12. The method of any one of claims 1-3, wherein the agent reacts with hypoxic cells and not mature blood cells, such that the mature blood cells are maintained.

13. The method of any one of claims 1-3, wherein the agent is selected from the group consisting of a bioreductive agent and a hypoxia-activated prodrug.

14. (canceled)

15. The method of claim 13 wherein the agent is Tirapazamine (TPZ).

16. The method of claim 13 wherein the agent is selected from the group consisting of: a benzotriazine, a nitroaromatic compound, an anthraquinone, a chloroquinoline DNA-targeting unit to 2-nitroimidazole, a dinitrobenzamide mustard, a nitrobenzyl phosphoramidate mustard, a nitroheterocyclic methylquaternary salt, a cobalt (III) complex and an indoloquinone.

17. The method of any one of claims 1-3, wherein the agent is selected from the group consisting of: misonidazole, RB 6145, AQ4N, NLCQ-1, SN 23862, and SN 28343.

18. The method of any one of claims 1-3, wherein the agent is an HIF-1α inhibitor selected from the group consisting of a camptothecin analogue, a topoisomerase (Topo)-I inhibitor, 3-(5′-hydroxymethyl-2′furyl)-1-benzyl indazole (YC-1) and PX-478.

19. (canceled)

20. The method of any one of claims 1-3, wherein the hypoxic cells are selected from the group consisting of primitive hematopoietic stem cells and late forming Cobblestone Area-Forming Cells (CAFCs).

21. (canceled)

22. The method of any one of claims 1-3, wherein depletion is measured in vitro in a Cobblestone Area-Forming Cells (CAFC) assay or in vivo by long-term engraftment of donor bone marrow transplanted in a subject.

23. (canceled)

24. The method of any one of claims 1-3, wherein the bone marrow is irradiated or contacted with a chemotherapeutic agent prior to or following contacting the cells with the agent.

25. The method of claim 2 or 3, wherein the host subject is administered a chemotherapeutic agent or irradiation prior to or following administration of the agent.

26. The method of any one of claims 1-3, wherein the hematopoietic stem cells from the donor subject are genetically modified.

27. The method of any one of claims 1-3, wherein the engrafting of donor hematopoietic stem cells is done in combination with a short-term immune modulating agent and wherein the short-term immune modulating agent is a T-cell depleting antibody.

28. (canceled)

29. Use of the method of any one of claims 1-3 to induce a state of donor-specific immune tolerance, to prevent or reduce graft-versus-host disease in a subject, to treat enzyme deficiency disease, to treat autoimmune diseases, or to treat a hematological cancer.

30-33. (canceled)
